SEQUENCE LISTING (102 sequences)

SEQ ID NO: 1 - protein, 9 aa, synthetic construct
  REGION 1..9: note = Amino Acids, artificial
YLQPRTFLL

SEQ ID NO: 2 - protein, 8 aa, synthetic construct [sequence omitted]

SEQ ID NO: 3 - protein, 13 aa, synthetic construct
  REGION 1..13: note = Amino Acids, artificial
CASGDENTGELFF

SEQ ID NO: 4 - protein, 13 aa, synthetic construct
  REGION 1..13: note = Amino Acids, artificial
CASSELNTGELFF

SEQ ID NO: 5 - protein, 11 aa, synthetic construct
  REGION 1..11: note = Amino Acids, artificial
CASSPEDTQYF

SEQ ID NO: 6 - protein, 13 aa, synthetic construct
  REGION 1..13: note = Amino Acids, artificial
CASSDLNTGELFF

SEQ ID NO: 7 - protein, 10 aa, synthetic construct
  REGION 1..10: note = Amino Acids, artificial
ASSLDIEAFF

SEQ ID NO: 8 - protein, 13 aa, synthetic construct
  REGION 1..13: note = Amino Acids, artificial
CASGEENTGELFF

SEQ ID NO: 9 - protein, 13 aa, synthetic construct
  REGION 1..13: note = Amino Acids, artificial
CASSSQNTGELFF

SEQ ID NO: 10 - protein, 11 aa, synthetic construct
  REGION 1..11: note = Amino Acids, artificial
CASSPDGEQYF

SEQ ID NO: 11 - protein, 13 aa, synthetic construct
  REGION 1..13: note = Amino Acids, artificial
CASSDDNTGELFF

SEQ ID NO: 12 - protein, 13 aa, synthetic construct
  REGION 1..13: note = Amino Acids, artificial
CSAGQRNTGELFF

SEQ ID NO: 13 - protein, 13 aa, synthetic construct
  REGION 1..13: note = Amino Acids, artificial
CATSEENTGELFF

SEQ ID NO: 14 - protein, 11 aa, synthetic construct
  REGION 1..11: note = Amino Acids, artificial
CASSPDIEAFF

SEQ ID NO: 15 - protein, 13 aa, synthetic construct
  REGION 1..13: note = Amino Acids, artificial
CASSYANTGELFF

SEQ ID NO: 16 - protein, 13 aa, synthetic construct
  REGION 1..13: note = Amino Acids, artificial
CATSRANTGELFF

SEQ ID NO: 17 - protein, 13 aa, synthetic construct
  REGION 1..13: note = Amino Acids, artificial
CASSQANTGELFF

SEQ ID NO: 18 - protein, 13 aa, synthetic construct
  REGION 1..13: note = Amino Acids, artificial
CASSDANTGELFF

SEQ ID NO: 19 - protein, 13 aa, synthetic construct
  REGION 1..13: note = Amino Acids, artificial
CASSEANTGELFF

SEQ ID NO: 20 - protein, 13 aa, synthetic construct
  REGION 1..13: note = Amino Acids, artificial
CASSLANTGELFF

SEQ ID NO: 21 - protein, 8 aa, synthetic construct [sequence omitted]

SEQ ID NO: 22 - protein, 8 aa, synthetic construct
  REGION 1..8: note = Amino Acids, artificial
CTTRGGYW

SEQ ID NO: 23 - protein, 18 aa, synthetic construct
  REGION 1..18: note = Amino Acids, artificial
CAKDPYYDFWSGYYFDYW

SEQ ID NO: 24 - protein, 8 aa, synthetic construct
  REGION 1..8: note = Amino Acids, artificial
CARGFDLW

SEQ ID NO: 25 - protein, 13 aa, synthetic construct
  REGION 1..13: note = Amino Acids, artificial
CARDRSGWYLDYW

SEQ ID NO: 26 - protein, 10 aa, synthetic construct [sequence omitted]

SEQ ID NO: 27 - protein, 18 aa, synthetic construct
  REGION 1..18: note = Amino Acids, artificial
CAKDPFYDFWSGYYFDYW

SEQ ID NO: 28 - protein, 18 aa, synthetic construct
  REGION 1..18: note = Amino Acids, artificial
CAKDPHYDFWSGYYFDYW

SEQ ID NO: 29 - protein, 13 aa, synthetic construct [sequence omitted]

SEQ ID NO: 30 - protein, 8 aa, synthetic construct [sequence omitted]

SEQ ID NO: 31 - protein, 7 aa, synthetic construct [sequence omitted]

SEQ ID NO: 32 - protein, 12 aa, synthetic construct
  REGION 1..12: note = Amino Acids, artificial
CARDDNWAFDYW

SEQ ID NO: 33 - protein, 12 aa, synthetic construct [sequence omitted]

SEQ ID NO: 34 - protein, 11 aa, synthetic construct [sequence omitted]

SEQ ID NO: 35 - protein, 12 aa, synthetic construct [sequence omitted]

SEQ ID NO: 36 - protein, 8 aa, synthetic construct [sequence omitted]

SEQ ID NO: 37 - protein, 10 aa, synthetic construct
  REGION 1..10: note = Amino Acids, artificial
CARDRADDYW

SEQ ID NO: 38 - protein, 17 aa, synthetic construct [sequence omitted]

SEQ ID NO: 39 - protein, 12 aa, synthetic construct [sequence omitted]

SEQ ID NO: 40 - protein, 12 aa, synthetic construct [sequence omitted]

SEQ ID NO: 41 - protein, 16 aa, synthetic construct
  REGION 1..16: note = Amino Acids, artificial
CAREGIVGATTGFDYW

SEQ ID NO: 42 - protein, 7 aa, synthetic construct [sequence omitted]

SEQ ID NO: 43 - protein, 16 aa, synthetic construct [sequence omitted]

SEQ ID NO: 44 - protein, 11 aa, synthetic construct
  REGION 1..11: note = Amino Acids, artificial
CARGADGMDVW

SEQ ID NO: 45 - protein, 7 aa, synthetic construct [sequence omitted]

SEQ ID NO: 46 - protein, 11 aa, synthetic construct
  REGION 1..11: note = Amino Acids, artificial
CARGHYGRDYW

SEQ ID NO: 47 - protein, 11 aa, synthetic construct [sequence omitted]

SEQ ID NO: 48 - protein, 16 aa, synthetic construct [sequence omitted]

SEQ ID NO: 49 - protein, 19 aa, synthetic construct [sequence omitted]

SEQ ID NO: 50 - protein, 19 aa, synthetic construct [sequence omitted]

SEQ ID NO: 51 - protein, 11 aa, synthetic construct [sequence omitted]

SEQ ID NO: 52 - protein, 15 aa, synthetic construct
  REGION 1..15: note = Amino Acids, artificial
CAKDLGYSSGLFDYW

SEQ ID NO: 53 - protein, 15 aa, synthetic construct
  REGION 1..15: note = Amino Acids, artificial
CAKDRGYYYYGMDVW

SEQ ID NO: 54 - protein, 13 aa, synthetic construct [sequence omitted]

SEQ ID NO: 55 - protein, 11 aa, synthetic construct
  REGION 1..11: note = Amino Acids, artificial
CARHFDWFDPW

SEQ ID NO: 56 - protein, 10 aa, synthetic construct
  REGION 1..10: note = Amino Acids, artificial
CARRGAFDLW

SEQ ID NO: 57 - protein, 18 aa, synthetic construct
  REGION 1..18: note = Amino Acids, artificial
CARARLTMLVVVDAFDLW

SEQ ID NO: 58 - protein, 18 aa, synthetic construct
  REGION 1..18: note = Amino Acids, artificial
CARARLTMLVVVGAFDLW

SEQ ID NO: 59 - protein, 12 aa, synthetic construct [sequence omitted]

SEQ ID NO: 60 - protein, 11 aa, synthetic construct [sequence omitted]

SEQ ID NO: 61 - protein, 14 aa, synthetic construct
  REGION 1..14: note = Amino Acids, artificial
CAKGGWKGDYFDFW

SEQ ID NO: 62 - protein, 11 aa, synthetic construct [sequence omitted]

SEQ ID NO: 63 - protein, 9 aa, synthetic construct [sequence omitted]

SEQ ID NO: 64 - protein, 5 aa, synthetic construct
  REGION 1..5: note = Amino Acids, artificial
CARDF

SEQ ID NO: 65 - protein, 8 aa, synthetic construct [sequence omitted]

SEQ ID NO: 66 - protein, 8 aa, synthetic construct [sequence omitted]

SEQ ID NO: 67 - protein, 12 aa, synthetic construct [sequence omitted]

SEQ ID NO: 68 - protein, 10 aa, synthetic construct [sequence omitted]

SEQ ID NO: 69 - protein, 9 aa, synthetic construct
  REGION 1..9: note = Amino Acids, artificial
CARDAFDLW

SEQ ID NO: 70 - protein, 11 aa, synthetic construct [sequence omitted]

SEQ ID NO: 71 - protein, 13 aa, synthetic construct [sequence omitted]

SEQ ID NO: 72 - protein, 11 aa, synthetic construct [sequence omitted]

SEQ ID NO: 73 - protein, 11 aa, synthetic construct [sequence omitted]

SEQ ID NO: 74 - protein, 10 aa, synthetic construct [sequence omitted]

SEQ ID NO: 75 - protein, 8 aa, synthetic construct [sequence omitted]

SEQ ID NO: 76 - protein, 4 aa, synthetic construct
  REGION 1..4: note = Amino Acids, artificial
CARW

SEQ ID NO: 77 - protein, 9 aa, synthetic construct [sequence omitted]

SEQ ID NO: 78 - protein, 8 aa, synthetic construct [sequence omitted]

SEQ ID NO: 79 - protein, 9 aa, synthetic construct [sequence omitted]

SEQ ID NO: 80 - protein, 11 aa, synthetic construct [sequence omitted]

SEQ ID NO: 81 - protein, 12 aa, synthetic construct
  REGION 1..12: note = Amino Acids, artificial
CARDQENWFDPW

SEQ ID NO: 82 - protein, 13 aa, synthetic construct [sequence omitted]

SEQ ID NO: 83 - protein, 14 aa, synthetic construct [sequence omitted]

SEQ ID NO: 84 - protein, 13 aa, synthetic construct [sequence omitted]

SEQ ID NO: 85 - protein, 13 aa, synthetic construct [sequence omitted]

SEQ ID NO: 86 - protein, 14 aa, synthetic construct [sequence omitted]

SEQ ID NO: 87 - protein, 15 aa, synthetic construct
  REGION 1..15: note = Amino Acids, artificial
CARDYDFWSGPFDYW

SEQ ID NO: 88 - protein, 15 aa, synthetic construct [sequence omitted]

SEQ ID NO: 89 - protein, 10 aa, synthetic construct [sequence omitted]

SEQ ID NO: 90 - protein, 7 aa, synthetic construct [sequence omitted]

SEQ ID NO: 91 - protein, 10 aa, synthetic construct [sequence omitted]

SEQ ID NO: 92 - protein, 10 aa, synthetic construct [sequence omitted]

SEQ ID NO: 93 - protein, 11 aa, synthetic construct
  REGION 1..11: note = Amino Acids, artificial
CATGGYYFDFW

SEQ ID NO: 94 - protein, 11 aa, synthetic construct
  REGION 1..11: note = Amino Acids, artificial
CSTGGYFFDYW

SEQ ID NO: 95 - protein, 8 aa, synthetic construct [sequence omitted]

SEQ ID NO: 96 - protein, 7 aa, synthetic construct [sequence omitted]

SEQ ID NO: 97 - protein, 10 aa, synthetic construct [sequence omitted]

SEQ ID NO: 98 - protein, 8 aa, synthetic construct
  REGION 1..8: note = Amino Acids, artificial
CTTVDGYW

SEQ ID NO: 99 - protein, 10 aa, synthetic construct [sequence omitted]

SEQ ID NO: 100 - protein, 17 aa, synthetic construct
  REGION 1..17: note = Amino Acids, artificial
CAKDRGYSSGWYYFDYW

SEQ ID NO: 101 - protein, 18 aa, synthetic construct
  REGION 1..18: note = Amino Acids, artificial
CAKDYYDSSGYPYYFDYW

SEQ ID NO: 102 - protein, 8 aa, synthetic construct [sequence omitted]

(19) United States
(12) Patent Application Publication
(10) Pub. No.: US 2024/0287606 A1
(43) Pub. Date: Aug. 29, 2024

(54) IMMUME CELL COUNTING BASED ON IMMUNE REPERTOIRE SEQUENCING

(71) Applicant: Roche Sequencing Solutions, Inc., Pleasanton, CA (US)

(72) Inventors: Jan Berka, Pleasanton, CA (US); Richard Dannebaum, Pleasant Hill, CA (US); Khai Luong, Oakland, CA (US); Florian Rubelt, Menlo Park, CA (US); Dilduz Telman, Sunnyvale, CA (US)

(21) Appl. No.: 18/475,498

(22) Filed: Sep. 27, 2023

Related U.S. Application Data
(63) Continuation of application No. PCT/EP2022/058362, filed on Mar. 30, 2022.
(60) Provisional application No. 63/169,802, filed on Apr. 1, 2021.

(51) Int. Cl. C12Q 1/6881

(52) U.S. Cl. CPC C12Q 1/6881

(57) ABSTRACT

The disclosure includes methods and compositions for accurately detecting subject's immune cell repertoire based on sequencing genomic DNA of immune cells.

### CROSS REFERENCE TO RELATED APPLICATIONS

This patent application is a continuation of International Patent Application No. PCT/EP2022/058362, filed Mar. 30, 2022, which claims priority to and the benefit of U.S. Patent Application No. 63/169,802, filed Apr. 1, 2021. Each of the above patent applications is incorporated herein by reference as if set forth in its entirety.

### SEQUENCE LISTING INCORPORATION BY REFERENCE

This application contains a sequence listing which has been filed electronically in xml format and is herein incorporated by reference in its entirety. Said xml copy has a file name of P36820-US1_sequence_listing.xml, was created on Sep. 21, 2023, and is 126 KB in size.

### FIELD OF THE DISCLOSURE

The disclosure relates to the field of immunology and more specifically, to assessing immune cells by sequencing immune gene sequences.

### BACKGROUND OF THE DISCLOSURE

The outbreak of COVID-19 caused by severe acute respiratory syndrome coronavirus 2 (SARS-CoV-2) presents a great threat to the current global public health due to its rapid transmission and high mortality rates. The global threat of SARS-CoV-2 lies in the life-threatening potential of COVID-19 for which it is the etiologic agent. SARS-CoV-2 has resulted in the global pandemic of COVID-19 that causes severe disease most often in adults and elderly individuals with comorbidities. In 2020 alone, COVID-19 is estimated to have killed almost 2 million persons worldwide (World Health Organization, 2020). Moreover, SARS1 and MERS coronaviruses have also caused severe disease in humans, while other coronaviruses have caused lethal infections in farm and companion animals.

T-cell receptor chains α, β, γ and δ are present in various amount in each person's immune repertoire. Changes in immune cell repertoire (i.e., the relative amount of immune cell types and immune cell clones) correlates with disease states and predisposition or susceptibility to disease. Measuring immune cell repertoire finds diagnostic applications in infectious disease and oncology. For instance, high-sensitivity detection of malignant immune cell clones allows for early detection of hematologic cancers as well as for treatment monitoring, and detection of minimal residual disease (MRD). Likewise, measuring immune cell repertoire facilitates the assessment of adaptive immunity including response to vaccination.

### SUMMARY OF THE DISCLOSURE

It is believed that changes in immune cell repertoire correlate with outcome of infection with SARS-CoV-2, including the outcome of COVID-19. As such, there exists a need to better understand changes immune cell repertoire as it relates to infection with SARS-CoV-2. In addition, there exists a need to better understand whether the host response to SARS-CoV-2 infection will be directly beneficial in managing COVID-19 and in preparing for the possible emergence of other pathogenic coronaviruses.

In view of the foregoing, the present disclosure provides a method of simultaneously determining a repertoire of T-cells and B-cells in a sample derived from a subject by detecting immune gene sequences in the T-cells and B-cells by a method comprising: a) contacting the sample with a plurality of immune cell receptor V gene specific primers, each primer including from 5′ to 3′: [5′-Phos], [SPLINT1], [BARCODE], and [V], wherein: [5′-Phos] is a 5′ phosphate; [SPLINT] is a first adaptor sequence; [BARCODE] is a unique molecular identifier barcode; and [V] is a sequence capable of hybridizing to an immune cell receptor V gene; b) hybridizing and extending the V gene specific primers to form a plurality of first double-stranded primer extension products; c) contacting the sample with an exonuclease to remove unhybridized V gene specific primers from the first double stranded primer extension products; d) contacting the sample with a plurality of immune cell receptor J gene specific primers, each primer including from 5′ to 3′: [5′-Phos], [SPLINT2], and [J], wherein: [5′-Phos] is a 5′ phosphate; [SPLINT2] is a second adaptor sequence; and [J] is a sequence capable of hybridizing to an immune cell receptor J gene; and further contacting the sample with a first universal primer capable of hybridizing to the first adaptor sequence; e) hybridizing and extending the J gene specific primers and the first universal primer to form a plurality of second double-stranded primer extension products; f) contacting the sample with an exonuclease to remove unhybridized J gene specific primers and first universal primer from the second double-stranded primer extension products; g) contacting the sample with first and second universal primers capable of hybridizing to the first and second adaptor sequences; h) amplifying the plurality of second double-stranded primer extension products; i) sequencing the amplified products to determine the immune gene sequences; j) grouping the determined immune gene sequences having the same unique molecular identified barcode (UMI) and the same complementarity determining region 3 (CDR3) into groups, each group representing a single immune cell; and k) determining a consensus within the groups thereby determining the repertoire of T-cells and B-cells in the sample. In some embodiments, the at least 20,000 unique CDR3 sequences are identified in step j).

In some embodiments, the [SPLINT] consists of 6 consecutive nucleotides, preferably of the sequence CGA TCT. In some embodiments, [BARCODE] consists of thirteen consecutive nucleotides selected from the group consisting of N and W, preferably of the sequence WNN NNN WNN NNN W.

In some embodiments, the V gene primers comprise a combination of VH (immunoglobulin) gene primers selected from the group consisting of Vα, Vβ, Vγ, and Vδ, gene primers. In some embodiments, the J gene primers comprise a combination of JH (immunoglobulin) gene primers selected from the group consisting of Jα, Jβ, Jγ, and Jδ primers. In some embodiments, only immune gene sequences representing productive rearrangements are used in determining the patient's repertoire of immune cells.

In some embodiments, the sample comprises cells separated from blood plasma. In some embodiments, the sample comprises captured CD4+ cells. In some embodiments, the sample comprises captured CD4+ cells and CD8+ cells.

In some embodiments, the repertoire of T-cells and B-cells comprises a CD4+ αβ T cell repertoire, a CD8+ αβ T cell repertoire, a B cell repertoire, a Vδ2+ T cell repertoire, and a Vδ1+ T cell repertoire. In some embodiments, the immune gene sequences in the T-cells comprise T-cell receptor (TCR) sequences. In some embodiments, the method further comprises measuring a quantity of the TCR sequences. In some embodiments, the TCR sequences comprise TRD sequences, TRB sequences, or a combination of TRD and TRB sequences.

In some embodiments, the immune gene sequences in the B-cells comprise B-cell receptor (BCR) sequences. In some embodiments, the method further comprises measuring a quantity of the BCR sequences. In some embodiments, the BCR sequences comprise IgH sequences.

In some embodiments, the method further comprises determining a ratio of the measured quantity of the TCR sequence to the measured quantity of the BCR sequences. In some embodiments, the method further comprises measuring immune cell repertoire diversity. In some embodiments, the method further comprises measuring immune cell repertoire focusing.

In some embodiments, the hybridizing in steps b) and/or e) comprises one or more cycles of a step-wise temperature drop of two or more steps. In some embodiments, the hybridizing in steps b) and/or e) comprises 20 cycles of temperature change from 60° C. to 57.5° C. and to 55° C. In some embodiments, the exonuclease in steps c) and/or f) is thermolabile. In some embodiments, the exonuclease is Exonuclease I.

Another aspect of the present disclosure is a method of characterizing a subject's antigen receptor repertoires by simultaneously enriching a sample derived from the subject for a plurality of immune gene sequences, comprising: a) contacting a sample derived from the subject with a plurality of immune cell receptor V gene specific primers, each primer including from 5′ to 3′: [5′-Phos], [SPLINT1], [BARCODE], and [V], wherein: [5′-Phos] is a 5′ phosphate; [SPLINT] is a first adaptor sequence; [BARCODE] is a unique molecular identifier barcode (UMI); and [V] is a sequence capable of hybridizing to an immune cell receptor V gene in the sample; b) hybridizing and extending the V gene specific primers to form a plurality of first double-stranded primer extension products; c) contacting the sample with an exonuclease to remove unhybridized V gene specific primers from the first double stranded primer extension products; d) contacting the sample with a plurality of immune cell receptor J gene specific primers, each primer including from 5′ to 3′: [5′-Phos], [SPLINT2], and [J], wherein: [5′-Phos] is a 5′ phosphate; [SPLINT2] is a second adaptor sequence; and [J] is a sequence capable of hybridizing to an immune cell receptor J gene; and further contacting the sample with a first universal primer capable of hybridizing to the first adaptor sequence; e) hybridizing and extending the J gene specific primers and the first universal primer to form a plurality of second double-stranded primer extension products; f) contacting the sample with an exonuclease to remove unhybridized J gene specific primers and first universal primer from the second double-stranded primer extension products; g) contacting the sample with first and second universal primers capable of hybridizing to the first and second adaptor sequences; h) amplifying the plurality of second double-stranded primer extension products; and i) sequencing the amplified products to determine the plurality of immune gene sequences, wherein each determined immune gene sequence of the plurality of determined immune gene sequences that is associated with a UMI represents a different immune cell repertoire.

In some embodiments, the method further comprises comparing the different determined immune cell repertoires to control immune cell repertoire data, wherein a change in representation of an immune cell type in the subject's antigen receptor repertoire indicates a disease state. In some embodiments, the different determined immune cell repertoires are selected from the group consisting of a CD4+ αβ T cell repertoire, aCD8+ αβ T cell repertoire, a B cell repertoire, a Vδ2+ T cell repertoire, and a Vδ1+ T cell repertoire.

In some embodiments, the method further comprises computing one or more entropic and dominance measures for each of the different determined immune cell repertoires. In some embodiments, the method further comprises computing a Shannon entropy for each of the different determined immune cell repertoires. In some embodiments, the method further comprises computing a Simpson's dominance for each of the different determined immune cell repertoires. In some embodiments, the method further comprises clustering identified patterns of SARS-CoV-2-associated adaptive T cell responsiveness. In some embodiments, the method of clustering the identified patterns of SARS-CoV-2-associated adaptive T cell responsiveness comprises identifying one or more discrete MHC Class I and Class II alleles

In some embodiments, the sample comprises a harvested PBMC sample. In some embodiments, the hybridizing in steps b) and/or e) comprises one or more cycles of a step-wise temperature drop of two or more steps. In some embodiments, the hybridizing in steps b) and/or e) comprises 20 cycles of temperature change from 60° C. to 57.5° C. and to 55° C. In some embodiments, the exonuclease in steps c) and/or f) is thermolabile. In some embodiments, the exonuclease is Exonuclease I.

Another aspect of the present disclosure is a method of detecting an immune reaction to a superantigen in a subject comprising measuring the subject's immune cell repertoire by the method described above, wherein an increased number of VP T-cells indicates immune response to a superantigen. In some embodiments, the increase is measured by normalizing the number of VP T-cells against the amount of DNA in the sample. In some embodiments, the increase is measured by normalizing the number of Vβ T-cells against the number of cells in the sample.

Another aspect of the present disclosure is a method of assessing prevalence of mucosal associated invariant T-cells (MAIT) in a test subject comprising measuring, by the method described above, the immune cell repertoires in a test subject and in a control subject, and comparing the numbers of unique Vβ sequences associated with MAIT in the subject and in the control subject, thereby determining the prevalence of MAIT in the test subject. In some embodiments, the increase is measured by normalizing the number of Vβ T-cells against the amount of DNA in the sample. In some embodiments, the increase is measured by normalizing the number of Vβ T-cells against the number of cells in the sample.

Another aspect of the present disclosure is a method of determining pathogen-specific immune sequences comprising: determining a subject's immune cell repertoire by the method described above, and comparing determined immune cell repertoire in one or more subjects infected with a pathogen and one or more control subjects, determining at least one immune cell present in more than one infected subject but not in the control subjects, thereby determining pathogen-specific immune cell sequences. In some embodiments, the immune gene sequences in the T-cells in the sample derived from the subject the T-cells comprise T-cell receptor (TCR) sequences, and wherein the pathogen-specific immune sequences include one or more TCRs. In some embodiments, the TCR sequences comprise TRD sequences, TRB sequences, or a combination of TRD and TRB sequences. In some embodiments, the immune gene sequences in the B cells of the sample derived from the subject comprise B-cell receptor (BCR) sequences, and wherein the pathogen-specific immune sequences include one or more BCRs. In some embodiments, the BCR sequences comprise IgH sequences.

Another aspect of the present disclosure is a method of detecting immune cell loss in a subject comprising: determining a subject's immune cell repertoire by the method described above, and comparing the determined immune cell repertoire in the subject and the control immune cell repertoires derived from one or more control subjects, identifying immune sequences present at a reduced level in the subject compared to the control subjects thereby detecting immune cell loss. In some embodiments, the immune gene sequences in the T-cells in the sample derived from the subject the T-cells comprise T-cell receptor (TCR) sequences. In some embodiments, the TCR sequences comprise TRD sequences, TRB sequences, or a combination of TRD and TRB sequences. In some embodiments, the immune gene sequences in the B cells of the sample derived from the subject comprise B-cell receptor (BCR) sequences. In some embodiments, the BCR sequences comprise IgH sequences.

Another aspect of the present disclosure is a method of determining disease state in a subject by determining an immune cell repertoire by detecting immune gene sequences in the cells by a method comprising: a) contacting a sample derived from the subject with a plurality of immune cell receptor V gene specific primers, each primer including from 5′ to 3′: [5′-Phos], [SPLINT1], [BARCODE], and [V], wherein: [5′-Phos] is a 5′ phosphate; [SPLINT] is a first adaptor sequence; [BARCODE] is a unique molecular identifier barcode (UMI); and [V] is a sequence capable of hybridizing to an immune cell receptor V gene in the sample; b) hybridizing and extending the V gene specific primers to form a plurality of first double-stranded primer extension products; c) contacting the sample with an exonuclease to remove unhybridized V gene specific primers from the first double stranded primer extension products; d) contacting the sample with a plurality of immune cell receptor J gene specific primers, each primer including from 5′ to 3′: [5′-Phos], [SPLINT2], and [J], wherein: [5′-Phos] is a 5′ phosphate; [SPLINT2] is a second adaptor sequence; and [J] is a sequence capable of hybridizing to an immune cell receptor J gene; and further contacting the sample with a first universal primer capable of hybridizing to the first adaptor sequence; e) hybridizing and extending the J gene specific primers and the first universal primer to form a plurality of second double-stranded primer extension products; f) contacting the sample with an exonuclease to remove unhybridized J gene specific primers and first universal primer from the second double-stranded primer extension products; g) contacting the sample with first and second universal primers capable of hybridizing to the first and second adaptor sequences; h) amplifying the plurality of second double-stranded primer extension products; i) sequencing the amplified products to determine a plurality of immune gene sequences, wherein each determined immune gene sequence of the plurality of determined immune gene sequences that is associated with a UMI represents a different immune cell repertoire; and j) comparing the different determined immune cell repertoires to control immune cell repertoire data, wherein a change in representation of an immune cell type in the subject's antigen receptor repertoire indicates a disease state.

In some embodiments, the different determined immune cell repertoires comprise IgH, TRB, and TRD repertoires. In some embodiments, the different determined immune cell repertoires are selected from the group consisting of a CD4+ αβ T cell repertoire, aCD8+ αβ T cell repertoire, a B cell repertoire, a Vδ2+ T cell repertoire, and a Vδ1+ T cell repertoire. In some embodiments, the different immune cell repertoires are simultaneously determined. In some embodiments, the V gene primers comprise a combination of VH (immunoglobulin) gene primers selected from the group consisting of Vα, Vβ, Vγ, and Vδ, gene primers. In some embodiments, the J gene primers comprise a combination of JH (immunoglobulin) gene primers selected from the group consisting of Jα, Jβ, Jγ, and Jδ primers.

Another aspect of the present disclosure is a method of simultaneously characterizing antigen receptor repertoires of CD4+ and CD8+ αβ T cells, B cells, and Vδ2+ and Vδ1+ T cells in a sample derived from a subject infected with SARS-CoV-2 infection, comprising: a) contacting a sample derived from the subject with a plurality of immune cell receptor V gene specific primers, each primer including from 5′ to 3′: [5′-Phos], [SPLINT1], [BARCODE], and [V], wherein: [5′-Phos] is a 5′ phosphate; [SPLINT] is a first adaptor sequence; [BARCODE] is a unique molecular identifier barcode (UMI); and [V] is a sequence capable of hybridizing to an immune cell receptor V gene in the sample; b) hybridizing and extending the V gene specific primers to form a plurality of first double-stranded primer extension products; c) contacting the sample with an exonuclease to remove unhybridized V gene specific primers from the first double stranded primer extension products; d) contacting the sample with a plurality of immune cell receptor J gene specific primers, each primer including from 5′ to 3′: [5′-Phos], [SPLINT2], and [J], wherein: [5′-Phos] is a 5′ phosphate; [SPLINT2] is a second adaptor sequence; and [J] is a sequence capable of hybridizing to an immune cell receptor J gene; and further contacting the sample with a first universal primer capable of hybridizing to the first adaptor sequence; e) hybridizing and extending the J gene specific primers and the first universal primer to form a plurality of second double-stranded primer extension products; f) contacting the sample with an exonuclease to remove unhybridized J gene specific primers and first universal primer from the second double-stranded primer extension products; g) contacting the sample with first and second universal primers capable of hybridizing to the first and second adaptor sequences; h) amplifying the plurality of second double-stranded primer extension products; i) sequencing the amplified products to determine a plurality of immune gene sequences associated with the CD4+ and CD8+ αβ T cells, the B cells, and the Vδ2+ and Vδ1+ T cells, wherein each determined immune gene sequence of the plurality of determined immune gene sequences that is associated with a UMI represents a different immune cell repertoire. In some embodiments, the V gene and J gene primers include primers targeting known human IgVH, TCRβ, and TCRδ V and J genes. In some embodiments, the V gene primers comprise a combination of VH (immunoglobulin) gene primers selected from the group consisting of Vα, Vβ, Vγ, and Vδ, gene primers. In some embodiments, the J gene primers comprise a combination of JH (immunoglobulin) gene primers. In some embodiments, the average sequence read-lengths ranged from about 170 nucleotides to about 210 nucleotides. In some embodiments, non-productive V-D-J rearrangements were excluded. In some embodiments, artifactual hybrid sequences were excluded. In some embodiments, the method further comprises computing one or more entropic and dominance measures for each of the antigen receptor repertoires. In some embodiments, the method further comprises computing a Shannon entropy for each of the antigen receptor repertoires. In some embodiments, the method further comprises computing a Simpson's dominance for each of the antigen receptor repertoires. In some embodiments, the method further comprises clustering identified patterns of SARS-CoV-2-associated adaptive T cell responsiveness. In some embodiments, the method further comprises focusing the antigen receptor repertoires.

### BRIEF DESCRIPTION OF THE DRAWINGS

FIGS. 1A-1C demonstrate that Immuno-PETE enables efficient, high fidelity and quantitative recovery of TRB, IGH and TRD CDR3s from PBMCs in a single combined multiplexed PCR. Immuno-PETE enables efficient, high fidelity and quantitative recovery of TRB, IGH and TRD CDR3s from PBMCs in a single combined multiplexed PCR. FIG. 1A Summary of workflow for samples recruited into the present study. Not all donors had longitudinal blood sampling. Not all healthy control samples were run through the full COVID-IP Study (Laing et al., 2020) pipeline. All samples had SARS-CoV-2 serology data. FIG. 1B Recovery of CDR3s by Immuno-PETE. 500 ng of genomic DNA used for CD4+ library preparation and 1000 ng of genomic DNA used for CD4− library preparation. Bar=median. Mann-Whitney test. FIG. 1C Correlation between sequencing and flow cytometry. Percentage of TRB, IGH and TRD CDR3s recovered by sequencing in the CD4− fraction (y-axis) versus percentage of CD3+, CD19+ and TCRgd+ on flow cytometry (x-axis) of the same sample. Spearman correlation. Note that the color-code for each cohort is maintained throughout for all the results figures that follow and the criteria for cohorts detailed in methods.

FIGS. 2A-2F illustrate repertoire changes in CD4+ and CD8+ T cells in COVID-19. FIG. 2A illustrates sub-sampled (to 2400 cells) CD4+ TRB repertoire diversity assessed by Shannon entropy, D50 and Simpson's dominance. FIG. 2B shows sub-sampled (to 1200 cells) CD8+ TRB repertoire diversity assessed by Shannon entropy, D50 and Simpson's dominance plotted. Bar=median. Kruskal-Wallis test with post hoc Dunn's test against age matched sero(−) control, unadjusted p values shown. FIG. 2C illustrates CD8+ TRB V gene family use as proportion of the unique CDR3s (i.e. each unique CDR3 is treated equally regardless of clone size). Median value from 20 sub-samples (to 1200 cells) plotted. FIG. 2D PCA of TRB V gene family use in CD8+ T cells as a percentage of the unique repertoire. Lower abundance of MAIT cell associated TRB V genes (TRBV6-1, TRBV6-4, TRBV20-1, black arrows: PCA loading) distinguishes active COVID-19 from sero(−). FIG. 2E provides a heatmap showing overlap of previously described SARS-CoV-2 antigen-specific TCRs (Shomuradova et al., 2020) with the present dataset. 12 of 344 peptide specific sequences on HLA-A*02:01 background from the study by Shomuradova et al shown here. Overlap was present in 2 of 8 individuals with active COVID-19, 4 of 9 sero(+) individuals and 0 of 17 sero(−) individuals (indicated by shaded cell). Color scale indicates proportion of total CD8 repertoire in each sample occupied by overlapped clones. FIG. 2F Defining SARS-CoV-2 associated TCR clusters. Circles represent individuals (identified by numbers) and exposure to SARS-CoV-2 (color). Clusters are built from all TCRs present in individuals with a specific HLA gene, in this example HLA B*07:02, by grouping similar CDR3s. We test each of such raw clusters for overrepresentation of SARS-CoV-2 exposed [active COVID-19 and sero(+)] or sero(−) individuals by Fisher's exact test (significance set at p<0.05). A representative cluster built from CD8+ TCRs of 1 sero(−) individual and 6 SARS-CoV-2 exposed individuals on an HLA B*07:02 background is shown.

FIGS. 3A-3E illustrate age-related focusing of Vd1 repertoire in SARS-CoV-2 exposed donors. FIG. 3(A) Overall TRDV1 repertoire diversity assessed by Shannon entropy, D50 and Simpson's dominance. Bar=median. Kruskal-Wallis test with post hoc Dunn's test against age matched sero(−) control, unadjusted p values shown. FIG. 3(B) Representative tree maps of TRDV1 repertoire from two seronegative donors aged ≥50 (left panels) and two donors aged ≥50 with active COVID-19 (right panels). Each circle represents a unique clone and the size of the circle is proportional to the size of the clone (numbers=clone count). FIG. 3(C) TRDV1 repertoire focusing assessed by Shannon entropy in individuals aged ≥50 exposed to SARS-CoV-2 plotted by severity of disease. Bar=median. Kruskal-Wallis test with post hoc Dunn's test against age matched sero(−) control, unadjusted p values shown. FIG. 3(D) Correlation of TRDV1 repertoire focusing (loss of Shannon entropy) and absolute numbers of CD45RA+/CD27− Vd1 cells per milliliter of blood assayed by the COVID-IP Study. Only samples with >30 TRDV1 CDR3s were analyzed for repertoire diversity and plotted. Spearman correlation. FIG. 3(E) Correlation of TRDV1 repertoire focusing (SE: Shannon entropy, D50, SD: Simpson's dominance) with CD4+ and CD8+ TRB focusing. Only samples with >30 TRDV1 CDR3s were analyzed for repertoire diversity and plotted. Color scale denotes Spearman r, significant correlations indicted with asterisk(s).

FIGS. 4A-4C illustrate age related selective depletion of Vd2 T cells in SARS-CoV-2 exposed donors. FIG. 4(A) Vd2 T cells (TRDV2) as a percentage of total gd T cells (TRDV). FIG. 4(B) d97-LVI pAg-reactive Vd2 T cells as a percentage of total Vd2 cells. FIG. 4(C) d97-LVI pAg-reactive Vd2 T cells as a percentage of total Vd2 cells plotted by severity of COVID-19 disease. Bar=median. Kruskal-Wallis test with post hoc Dunn's test against age matched sero(−) control, unadjusted p values shown.

FIGS. 5A-5C show overall IGH repertoire diversity assessed by Shannon entropy, D50 and Simpson's dominance. FIG. 5A Overall IGH repertoire diversity assessed by Shannon entropy, D50 and Simpson's dominance. FIG. 5B Within cluster IGH repertoire diversity assessed by Shannon entropy, D50 and Simpson's dominance. Bar=median. Kruskal-Wallis test with post hoc Dunn's test against age matched sero(−) control, unadjusted p values shown. FIG. 5C IGH repertoire focusing within clusters assessed by Shannon entropy (left) as well as normalized anti-Spike IgM titres (right) plotted by time from symptom onset (1-week bins).

FIGS. A-F show average clone count per sample of SARS-CoV-2 enriched CDR3 sequences. FIG. 6A sets forth average clone count per sample of SARS-CoV-2 enriched CDR3 matches grouped by disease status: active COVID-19 (n=40 samples), sero(+) (n=15 samples) or sero(−) (n=9 samples). Kruskal-Wallis with post hoc Dunn's test. FIG. 6(B) Average clone count per sample of seronegative enriched CDR3 matches grouped by disease status: active COVID-19 (n=9 samples), sero(+) (n=8 samples) or sero(−) (n=45 samples). Kruskal-Wallis with post hoc Dunn's test. FIG. 6(C) Average clone fraction per sample of SARS-CoV-2 enriched CDR3 matches, excluding singleton matches, grouped by disease status: active COVID-19 (n=22 samples), sero(+) (n=9 samples). No non-singleton matches were detected in the sero(−) cohort for the SARS-CoV-2 enriched sequences and so this group is not included. Mann-Whitney test. FIG. 6(D) Sum of clone fractions of SARS-CoV-2 enriched CDR3 matches per sample, excluding singleton matches, grouped by disease status: active COVID-19 (n=22 samples), sero(+) (n=9 samples). No non-singleton matches were detected in the seronegative cohort for the SARS-CoV-2 enriched sequences and so this group is not included). Mann-Whitney test. FIG. 6(E) Frequency of more than one nucleotide sequence encoding a unique amino acid IGH CDR3 sequence in SARS-CoV-2 enriched AA CDR3 matches. Fisher's exact test. FIG. 6(F) Heatmap showing correlations of the sum of clone fraction of SARS-CoV-2 enriched CDR3 matches and related clustered sequences with days from symptom onset, global IGH repertoire diversity, B cell populations and SARS-CoV-2 serology. Color scale denotes Spearman r, only significant correlations are colored.

FIGS. 7A-C show results related to age in years of donors at the time of baseline blood sampling. FIG. 7(A) Age in years of donors at the time of baseline blood sampling. Bar=median. Kruskal-Wallis followed by post-hoc Dunn's test corrected for multiple testing. FIG. 7(B) Summary of gender proportions in study cohorts. FIG. 7(C) Summary of Immuno-PETE workflow. Genomic DNA is selectively amplified in a multiplex PCR reaction with UMI tagged primers for all known TRBV, TRBJ, IGHV, IGHJ, TRDV and TRDJ genes. Clustered reads based on identical (or near identical) UMI+CDR3 reads corrects for PCR errors (*) and amplification bias allowing for quantitative mapping to input cells.

FIGS. 8A-F show overall CD4+ TRB repertoire diversity assessed by Shannon entropy and Simpson's dominance. FIG. 8(A) Overall CD4+ TRB repertoire diversity assessed by Shannon entropy and Simpson's dominance. FIG. 8(B) Overall CD8+ TRB repertoire diversity assessed by Shannon entropy and Simpson's dominance. Bar=median. Kruskal-Wallis test with post hoc Dunn's test against age matched sero(−) control, unadjusted p values shown. FIG. 8(C) Correlation of overall CD4+ TRB (top) or CD8+ TRB (bottom) diversity with frequency of naïve CD45RA+CCR7+CD4+ or CD8+ T cells as previously reported in the COVID-IP study. Spearman correlation. FIG. 8(D) CD4+ TRB V gene family use as proportion of the unique CDR3s (i.e. each unique CDR3 is treated equally regardless of clone size). Median value from 20 sub-samples (to 2400 cells) plotted. FIG. 8(E) Frequency of more than one nucleotide sequence encoding a unique amino acid CDR3 in SARS-CoV-2 exposed enriched clusters versus the entire repertoire in CD4+(left) and CD8+(right) cells. Fisher's exact test. FIG. 8(F) Fraction of unique TCRs in clusters in CD4+ cells. Bar=median. Kruskal-Wallis test with post hoc Dunn's test against age matched sero(−) control, unadjusted p values shown.

FIGS. 9A-C show amino acid (AA) CDR3 lengths of unique Vd1 CDR3s plotted as a percentage of total unique Vd1 CDR3s. Amino acid (AA) CDR3 lengths of unique Vd1 CDR3s plotted as a percentage of total unique Vd1 CDR3s. Each point represents one sample. FIG. 9A: sero(−) samples. FIG. 9B: SARS-CoV-2 exposed samples (sero(+) and active COVID-19 cohort). FIG. 9C: data from previous study of breast tumor infiltrating Vd1 T cells from donors with triple-negative breast cancer (TNBC). Only samples with >30 Vd1 CDR3s were analyzed for CDR3 length and plotted.

FIGS. 10A-10C shows overall Vd2 repertoire diversity assessed by Shannon entropy, D50 and Simpson's dominance. Bar=median. Kruskal-Wallis test with post hoc Dunn's test against age matched sero(−) control, unadjusted p values shown. Only samples with >30 Vd2 CDR3s were analyzed for repertoire diversity and plotted. Overall Vd2 repertoire diversity assessed by Shannon entropy, D50 and Simpson's dominance. Bar=median. Kruskal-Wallis test with post hoc Dunn's test against age matched sero(−) control, unadjusted p values shown. Only samples with >30 Vd2 CDR3s were analyzed for repertoire diversity and plotted.

FIGS. 11A-B show correlation of IGH repertoire focusing with CD4+ and CD8+ TRB and TRDV1 focusing. FIG. 11 (A) Correlation of IGH repertoire focusing (SE: Shannon entropy, D50, SD: Simpson's dominance) with CD4+ and CD8+ TRB and TRDV1 focusing. Only samples with >30 TRDV1 CDR3s were analyzed for repertoire diversity and plotted. Color scale denotes Spearman r, significant correlations indicted with asterisk(s). FIG. 11 (B) IgH CDR3s clustered as a proportion of total IgH CDR3s. Bar=median. Kruskal-Wallis test with post hoc Dunn's test against age matched sero(−) control, unadjusted p values shown.

FIGS. 12A-E display an algorithm for generating an initial list of 318 IgH CDR3 sequences shared by at least 3 donors in across the study. FIG. 12(A) Algorithm for generating an initial list of 318 IgH CDR3 sequences shared by at least 3 donors in across the study (*excluding those exclusively shared amongst the sero(+) cohort). FIG. 12(B) Algorithm for selecting SARS-CoV-2 exposed enriched IgH CDR3 sequences. FIG. 12(C) Algorithm for selecting sero(−) enriched IgH CDR3 sequences. FIG. 12(D) Average clone count of SARS-CoV-2 enriched IgH CDR3s found per sample plotted against total B cells recovered per sample demonstrates no correlation. FIG. 12 (E) Proportion of the 41 SARS-CoV-2 enriched IgH CDR3 sequences which were also found in clonally related clusters identified using the Change-O clustering algorithm (n=24/41).

FIG. 13 lists the 41 SARS-CoV-2 enriched sequences ordered by number of SARS-CoV-2 exposed subjects with matches for the sequence. Concordance between presence in clusters and clonal expansion indicated by orange shading in last two columns. The table within FIG. 13 lists the 41 SARS-CoV-2 enriched sequences ordered by number of SARS-CoV-2 exposed subjects with matches for the sequence. Concordance between presence in clusters and clonal expansion indicated by orange shading in last two columns.

FIG. 14 lists the 53 sero(−) enriched sequences ordered by number sero(−) subjects with matches for the sequence. The table within FIG. 14 lists the 53 sero(−) enriched sequences ordered by number sero(−) subjects with matches for the sequence. None of these sequences were identified as significantly enriched in any cohort in the iReceptor database or found in the COV-Ab-Dab database of SARS-CoV-2-reactive sequences. Concordance between presence in clusters and clonal expansion indicated by orange shading in last two columns.

### DETAILED DESCRIPTION OF THE DISCLOSURE

It should also be understood that, unless clearly indicated to the contrary, in any methods claimed herein that include more than one step or act, the order of the steps or acts of the method is not necessarily limited to the order in which the steps or acts of the method are recited.

### Definitions

As used herein, the singular terms “a,” “an,” and “the” include plural referents unless context clearly indicates otherwise. Similarly, the word “or” is intended to include “and” unless the context clearly indicates otherwise. The term “includes” is defined inclusively, such that “includes A or B” means including A, B, or A and B.

As used herein in the specification and in the claims, “or” should be understood to have the same meaning as “and/or” as defined above. For example, when separating items in a list, “or” or “and/or” shall be interpreted as being inclusive, e.g., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (e.g. “one or the other but not both”) when preceded by terms of exclusivity, such as “either,” “one of,” “only one of” or “exactly one of.” “Consisting essentially of,” when used in the claims, shall have its ordinary meaning as used in the field of patent law.

The terms “comprising,” “including,” “having,” and the like are used interchangeably and have the same meaning. Similarly, “comprises,” “includes,” “has,” and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a device having components a, b, and c” means that the device includes at least components a, b, and c. Similarly, the phrase: “a method involving steps a, b, and c” means that the method includes at least steps a, b, and c. Moreover, while the steps and processes may be outlined herein in a particular order, the skilled artisan will recognize that the ordering steps and processes may vary.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Reference throughout this specification to “one embodiment” or “an embodiment” means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used herein, the term “adapter” refers a nucleotide sequence that may be added to another sequence so as to import additional properties to that sequence. An adapter can be single- or double-stranded, or may have both a single-stranded portion and a double-stranded portion.

As used herein “amplification” refers to a process in which a copy number increases. Amplification may be a process in which replication occurs repeatedly over time to form multiple copies of a template. Amplification can produce an exponential or linear increase in the number of copies as amplification proceeds. Exemplary amplification strategies include polymerase chain reaction (PCR), loop-mediated isothermal amplification (LAMP), rolling circle replication (RCA), cascade-RCA, nucleic acid based amplification (NASBA), and the like. Also, amplification can utilize a linear or circular template. Amplification can be performed under any suitable temperature conditions, such as with thermal cycling or isothermally. Furthermore, amplification can be performed in an amplification mixture (or reagent mixture), which is any composition capable of amplifying a nucleic acid target, if any, in the mixture. PCR amplification relies on repeated cycles of heating and cooling (i.e., thermal cycling) to achieve successive rounds of replication. PCR can be performed by thermal cycling between two or more temperature setpoints, such as a higher denaturation temperature and a lower annealing/extension temperature, or among three or more temperature setpoints, such as a higher denaturation temperature, a lower annealing temperature, and an intermediate extension temperature, among others. PCR can be performed with a thermostable polymerase, such as Taq DNA polymerase. PCR generally produces an exponential increase in the amount of a product amplicon over successive cycles.

As used herein, the term “barcode” refers to a nucleic acid sequence that can be detected and identified. Barcodes can be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more nucleotides long. Barcodes can employ error-correcting codes such that one or more errors in synthesis, replication, and/or sequencing can be corrected to identify the barcode sequence. Examples of error correcting codes and their use in barcodes and barcode identification and/or sequencing include, but are not limited to, those described in U.S. 2010/0,323,348; and U.S. Pat. No. 8,715,967. In some cases, the barcodes are designed to have a minimum number of distinct nucleotides with respect to all other barcodes of a population. The minimum number can be 1, 2, 3, 4, 5, 6, 7, 8, 9, 10, 11, 12, 13, 14, 15, or more. Thus, for example, a population of barcodes having a minimum number of at least five distinct nucleotides will differ at least five nucleotide positions from all other barcodes in the population. Examples of barcodes, multiplex identifiers, or unique molecular identifiers are described in U.S. Publication No. 2020/0032244, and in U.S. Pat. Nos. 7,393,665, 8,168,385, 8,481,292, 8,685,678, and 8,722,368, and in PCT Publication No. WO/2018/138237, the disclosures of which are hereby incorporated by reference herein in their entireties.

As used herein, a “B cell receptor” or “BCR” refers to the secreted or membrane bound antigen recognition complex of a B cell. The BCR is composed of two different protein chains (e.g., heavy and light). Each chain contains a variable region (V), a joining region (J), and a constant region (C). The variable region contains hypervariable complementarity determining regions (CDRs). Heavy chains can further contain a diversity region (D) between the V and J regions. Further BCR diversity is generated by VJ (for light chains) and VDJ (for heavy chains) recombination as well as somatic hypermutation of recombined chains. The terms also refer to various recombinant and heterologous forms.

As used herein, the term “complementary” generally refers to the capability for precise pairing between two nucleotides. The term “complementary” refers to the ability to form favorable thermodynamic stability and specific pairing between the bases of two nucleotides at an appropriate temperature and ionic buffer conditions. Complementarity is achieved by distinct interactions between the nucleobases adenine, thymine (uracil in RNA), guanine and cytosine, where adenine pairs with thymine or uracil, and guanine pairs with cytosine. For example, if a nucleotide at a given position of a nucleic acid is capable of hydrogen bonding with a nucleotide of another nucleic acid, then the two nucleic acids are considered to be complementary to one another at that position. Complementarity between two single-stranded nucleic acid molecules may be “partial,” in which only some of the nucleotides bind, or it may be complete when total complementarity exists between the single-stranded molecules. A first nucleotide sequence can be said to be the “complement” of a second sequence if the first nucleotide sequence is complementary to the second nucleotide sequence. A first nucleotide sequence can be said to be the “reverse complement” of a second sequence, if the first nucleotide sequence is complementary to a sequence that is the reverse (i.e., the order of the nucleotides is reversed) of the second sequence.

As used herein, the term “enrichment” refers to the process of increasing the relative abundance of a population of molecules, e.g. nucleic acid molecules, in a sample relative to the total amount of the molecules initially present in the sample before treatment. Thus, an enrichment step provides a percentage or fractional increase rather than directly increasing for example, the copy number of the nucleic acid sequences of interest as amplification methods, such as a polymerase chain reaction, would.

The term “next generation sequencing” refers to sequencing technologies having high-throughput sequencing as compared to traditional Sanger- and capillary electrophoresis-based approaches, wherein the sequencing process is performed in parallel, for example producing thousands or millions of relatively small sequence reads at a time. Some examples of next generation sequencing techniques include, but are not limited to, sequencing by synthesis, sequencing by ligation, and sequencing by hybridization. These technologies produce shorter reads (anywhere from about 25-about 500 bp) but many hundreds of thousands or millions of reads in a relatively short time.

Examples of such sequencing devices available from Illumina (San Diego, CA) include, but are not limited to iSEQ, MiniSEQ, MiSEQ, NextSEQ, NoveSEQ. It is believed that the Illumina next-generation sequencing technology uses clonal amplification and sequencing by synthesis (SBS) chemistry to enable rapid sequencing. The process simultaneously identifies DNA bases while incorporating them into a nucleic acid chain. Each base emits a unique fluorescent signal as it is added to the growing strand, which is used to determine the order of the DNA sequence.

A non-limiting example of a sequencing device available from ThermoFisher Scientific (Waltham, MA) includes the Ion Personal Genome Machine™ (PGM™) System. It is believed that Ion Torrent sequencing measures the direct release of H+ (protons) from the incorporation of individual bases by DNA polymerase. A non-limiting example of a sequencing device available from Pacific Biosciences (Menlo Park, CA) includes the PacBio Sequel Systems. A non-limiting example of a sequencing device available from Roche (Pleasanton, CA) is the Roche 454.

Next-generation sequencing methods may also include nanopore sequencing methods. In general, three nanopore sequencing approaches have been pursued: strand sequencing in which the bases of DNA are identified as they pass sequentially through a nanopore, exonuclease-based nanopore sequencing in which nucleotides are enzymatically cleaved one-by-one from a DNA molecule and monitored as they are captured by and pass through the nanopore, and a nanopore sequencing by synthesis (SBS) approach in which identifiable polymer tags are attached to nucleotides and registered in nanopores during enzyme-catalyzed DNA synthesis. Common to all these methods is the need for precise control of the reaction rates so that each base is determined in order.

Strand sequencing requires a method for slowing down the passage of the DNA through the nanopore and decoding a plurality of bases within the channel; ratcheting approaches, taking advantage of molecular motors, have been developed for this purpose. Exonuclease-based sequencing requires the release of each nucleotide close enough to the pore to guarantee its capture and its transit through the pore at a rate slow enough to obtain a valid ionic current signal. In addition, both of these methods rely on distinctions among the four natural bases, two relatively similar purines and two similar pyrimidines. The nanopore SBS approach utilizes synthetic polymer tags attached to the nucleotides that are designed specifically to produce unique and readily distinguishable ionic current blockade signatures for sequence determination.

In some embodiments, sequencing of nucleic acids comprises via nanopore sequencing comprises: preparing nanopore sequencing complexes and determining polynucleotide sequences. Methods of preparing nanopores and nanopore sequencing are described in U.S. Patent Application Publication No. 2017/0268052, and PCT Publication Nos. WO2014/074727, WO2006/028508, WO2012/083249, and WO/2014/074727, the disclosures of which are hereby incorporated by reference herein in their entireties. In some embodiments, tagged nucleotides may be used in the determination of the polynucleotide sequences (see, e.g., PCT Publication No. WO/2020/131759, WO/2013/191793, and WO/2015/148402, the disclosures of which are hereby incorporated by reference herein in their entireties).

Analysis of the data generated by sequencing is generally performed using software and/or statistical algorithms that perform various data conversions, e.g., conversion of signal emissions into base calls, conversion of base calls into consensus sequences for a nucleic acid template, etc. Such software, statistical algorithms, and the use of such are described in detail, in U.S. Patent Application Publication Nos. 2009/0024331 2017/0044606 and in PCT Publication No. WO/2018/034745, the disclosures of which are hereby incorporated by reference herein in their entireties.

As used herein, the term “nucleic acid” refers to a polymeric form of nucleotides of any length, either deoxyribonucleotides or ribonucleotides, or analogs thereof. Polynucleotides may have any three-dimensional structure, and may perform any function, known or unknown. Unless specifically limited, the terms encompass nucleic acids or polynucleotides including known analogues of natural nucleotides that have similar binding properties as the reference nucleic acid and are metabolized in a manner similar to naturally occurring nucleotides. Non-limiting examples of polynucleotides include coding or non-coding regions of a gene or gene fragment, loci (locus) defined from linkage analysis, exons, introns, messenger RNA (mRNA), transfer RNA, ribosomal RNA, ribozymes, cDNA, synthetic polynucleotides, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified, such as by conjugation with a labeling component. Unless otherwise indicated, a particular nucleic acid sequence also implicitly encompasses conservatively modified variants thereof (e.g., degenerate codon substitutions), alleles, orthologues, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

As used herein, the term “polymerase” refers to an enzyme that performs template-directed synthesis of polynucleotides. A DNA polymerase can add free nucleotides only to the 3′ end of the newly forming strand. This results in elongation of the newly forming strand in a 5′-3′ direction. No known DNA polymerase is able to begin a new chain (de novo). DNA polymerase can add a nucleotide only on to a pre-existing 3′-OH group, and, therefore, needs a primer at which it can add the first nucleotide. Non-limiting examples of polymerases include prokaryotic DNA polymerases (e.g. Pol I, Pol II, Pol III, Pol IV and Pol V), eukaryotic DNA polymerase, archaeal DNA polymerase, telomerase, reverse transcriptase and RNA polymerase. Reverse transcriptase is an RNA-dependent DNA polymerase which synthesizes DNA from an RNA template. The reverse transcriptase family contain both DNA polymerase functionality and RNase H functionality, which degrades RNA base-paired to DNA. RNA polymerase is an enzyme that synthesizes RNA using DNA as a template during the process of gene transcription. RNA polymerase polymerizes ribonucleotides at the 3′ end of an RNA transcript.

In some embodiments, a polymerase from the following may be used in a polymerase-mediated primer extension, end-modification (e.g., terminal transferase, degradation, or polishing), or amplification reaction: archaea (e.g., Thermococcus litoralis (Vent, GenBank: AAA72101), Pyrococcus furiosus (Pfu, GenBank: D12983, BAA02362), Pyrococcus woesii, Pyrococcus GB-D (Deep Vent, GenBank: AAA67131), Thermococcus kodakaraensis KODI (KOD, GenBank: BD175553, BAA06142; Thermococcus sp. strain KOD (Pfx, GenBank: AAE68738)), Thermococcus gorgonarius (Tgo, Pdb: 4699806), Sulfolobus solataricus (GenBank: NC002754, P26811), Aeropyrum pernix (GenBank: BAA81109), Archaeglobus fulgidus (GenBank: 029753), Pyrobaculum aerophilum (GenBank: AAL63952), Pyrodictium occultum (GenBank: BAA07579, BAA07580), Thermococcus 9 degree Nm (GenBank: AAA88769, Q56366), Thermococcus fumicolans (GenBank: CAA93738, P74918), Thermococcus hydrothermalis (GenBank: CAC18555), Thermococcus sp. GE8 (GenBank: CAC12850), Thermococcus sp. JDF-3 (GenBank: AX135456; WO0132887), Thermococcus sp. TY (GenBank: CAA73475), Pyrococcus abyssi (GenBank: P77916), Pyrococcus glycovorans (GenBank: CAC12849), Pyrococcus horikoshii (GenBank: NP 143776), Pyrococcus sp. GE23 (GenBank: CAA90887), Pyrococcus sp. ST700 (GenBank: CAC 12847), Thermococcus pacificus (GenBank: AX411312.1), Thermococcus zilligii (GenBank: DQ3366890), Thermococcus aggregans, Thermococcus barossii, Thermococcus celer (GenBank: DD259850.1), Thermococcus profundus (GenBank: E14137), Thermococcus siculi (GenBank: DD259857.1), Thermococcus thioreducens, Thermococcus onnurineus NA1, Sulfolobus acidocaldarium, Sulfolobus tokodaii, Pyrobaculum calidifontis, Pyrobaculum islandicum (GenBank: AAF27815), Methanococcus jannaschii (GenBank: Q58295), Desulforococcus species TOK, Desulforococcus, Pyrolobus, Pyrodictium, Staphylothermus, Vulcanisaetta, Methanococcus (GenBank: P52025) and other archaeal B polymerases, such as GenBank AAC62712, P956901, BAAA07579)), thermophilic bacteria Thermus species (e.g., flavus, ruber, thermophilus, lacteus, rubens, aquaticus), Bacillus stearothermophilus, Thermotoga maritima, Methanothermus fervidus, KOD polymerase, TNA1 polymerase, Thermococcus sp. 9 degrees N-7, T4, T7, phi29, Pyrococcus furiosus, P. abyssi, T. gorgonarius, T. litoralis, T. zilligii, T. sp. GT, P. sp. GB-D, KOD, Pfu, T. gorgonarius, T. zilligii, T. litoralis and Thermococcus sp. 9N-7 polymerases.

As used herein, the term “primer” refers to an oligonucleotide which binds to a specific region of a single-stranded template nucleic acid molecule and initiates nucleic acid synthesis via a polymerase-mediated enzymatic reaction, extending from the 3′ end of the primer and complementary to the sequence of the template molecule. PCR amplification primers can be referred to as ‘forward’ and ‘reverse’ primers, one of which is complementary to a nucleic acid strand and the other of which is complementary to the complement of that strand. Typically, a primer comprises fewer than about 100 nucleotides and preferably comprises fewer than about 30 nucleotides. Exemplary primers range from about 5 to about 25 nucleotides. Primers can comprise, for example, RNA and/or DNA bases, as well as non-naturally occurring bases. The directionality of the newly forming strand (the daughter strand) is opposite to the direction in which DNA polymerase moves along the template strand. In some cases, a target capture primer specifically hybridizes to a target polynucleotide under hybridization conditions. Such hybridization conditions can include, but are not limited to, hybridization in isothermal amplification buffer (20 mM Tris-HCl, 10 mM (NH4)2SO4), 50 mM KCl, 2 mM MgSO4, 0.1% TWEEN® 20, pH 8.8 at 25° C.) at a temperature of about 40° C., 45° C., 50° C., 55° C., 60° C., 65° C., or 70° C.

As used herein the term “sample” refers to any biological sample that comprises nucleic acid molecules, typically comprising DNA and/or RNA. Samples may be tissues, cells or extracts thereof, or may be purified samples of nucleic acid molecules. Use of the term “sample” does not necessarily imply the presence of target sequence within nucleic acid molecules present in the sample. In some cases, the “sample” comprises immune cells (e.g., B cells and/or T cells), or a fraction thereof (e.g., a fraction enriched in genomic DNA, total RNA, or mRNA). In some embodiments, a sample can comprise a FACS sorted population of cells (such as human T cells) or a fixed formalin paraffin embedded (FFPE) tissue sample.

As used herein, the term “sequence,” when used in reference to a nucleic acid molecule, refers to the order of nucleotides (or bases) in the nucleic acid molecules. In cases, where different species of nucleotides are present in the nucleic acid molecule, the sequence includes an identification of the species of nucleotide (or base) at respective positions in the nucleic acid molecule. A sequence is a property of all or part of a nucleic acid molecule. The term can be used similarly to describe the order and positional identity of monomeric units in other polymers such as amino acid monomeric units of protein polymers.

As used herein, the term “sequencing” refers to the determination of the order and position of bases in a nucleic acid molecule. More particularly, the term “sequencing” refers to biochemical methods for determining the order of the nucleotide bases, adenine, guanine, cytosine, and thymine, in a DNA oligonucleotide. Sequencing, as the term is used herein, can include without limitation parallel sequencing or any other sequencing method known of those skilled in the art, for example, chain-termination methods, rapid DNA sequencing methods, wandering-spot analysis, Maxam-Gilbert sequencing, dye-terminator sequencing, or using any other modern automated DNA sequencing instruments.

As used herein, a “T cell receptor” or “TCR” refers to the antigen recognition complex of a T cell. The TCR is composed of two different protein chains (e.g., alpha and beta or gamma and delta). Each chain is composed of two extracellular domains containing a variable region (V), a joining region (J), and a constant region (C). The variable region contains hypervariable complementarity determining regions (CDRs). Beta and delta TCR chains further contain a diversity region (D) between the V and J regions. Further TCR diversity is generated by VJ (for alpha and gamma chains) and VDJ (for beta and delta chains) recombination. The terms also refer to various recombinant and heterologous forms, including soluble TCRs expressed from a heterologous system.

As used herein, the term “universal primer” refers to a primer that can hybridize to and support amplification of target polynucleotides having a shared complementary universal primer binding site. Similar, the term “universal primer pair” refers to a forward and reverse primer pair that can hybridize to and support PCR amplification of target polynucleotides having shared complementary forward and reverse universal primer binding sites. Such universal primer(s) and universal primer binding site(s) can allow single or double primer mediated universal amplification (e.g., universal PCR) of target polynucleotide regions of interest.

The headings provided herein are for convenience only and do not interpret the scope or meaning of the disclosed embodiments.

### Overview

Although precise correlates of protection vis-à-vis SARS-CoV-2 infection are not yet defined, it is reasonable to assume that the adaptive immune system plays a major role. For example, most SARS-CoV-2 infections are asymptomatic or pauci-symptomatic, particularly among younger people who have greater immunocompetence than the elderly (Lavezzo et al., 2020; Tabata et al., 2020). Moreover, despite there being over 100 million documented infections, incidences of reinfection are rare, even in a milieu of high virus transmission, consistent with primary infection driving the establishment of adaptive immune memory (Hansen et al., 2021). In addition, there is at least some evidence for the immune response to SARS-CoV-2 being influenced by memory responses to prior common cold coronavirus infections (Grifoni et al., 2020; Le Bert et al., 2020; Sokal et al., 2021). Furthermore, the efficacy of several types of SARS-CoV-2 vaccines seem to be associated with the development of robust B and T cell responses (Folegatti et al., 2020; Jackson et al., 2020; Sahin et al., 2020). In sum, a better characterization of the nature, and quality of anti-SARS-CoV-2 adaptive responses seems essential if the threat of COVID-19 is to be eliminated and better manage the risks posed by other coronaviruses.

Interestingly, many analyses of COVID-19 have linked disease severity to specific immune parameters. Thus, patients presenting with increasingly severe disease display markedly elevated levels of IL10, IP10, and IL6; dramatic peripheral blood depletions of basophils and plasmacytoid dendritic cells; altered monocyte differentiation profiles; and highly atypical peripheral blood populations of cycling myeloid and T cells (Laing et al., 2020). There is also an overt, concurrent activation, exhaustion, and depletion of selective T cell subsets, with consequential increases in neutrophil-to-lymphocyte ratios (Mathew et al., 2020; Song et al., 2020; Wilk et al., 2020; Zheng et al., 2020). The causes for these chaotic traits are mostly unknown, but some are shared with other overt disease settings, including sepsis and severe influenza.

In integrating these observations, COVID-19 offers a rare opportunity to investigate how human immunoprotective adaptive responses become established in response to a defined stimulus, against a backdrop of overt immunological dysregulation in bona fide life-threatening settings.

Adaptive immune receptor repertoire sequencing is a powerful tool to analyze the immune response in many diseases. Multiple methods exist to assess an immune gene repertoire. For example, an immune gene repertoire may be assessed using the methods described in U.S. Pat. No. 11,098,360, the disclosure of which is hereby incorporated by reference herein in its entirety.

The present disclosure utilizes a primer extension target enrichment (PETE) method that includes two flanking primers (hereinafter the “flanking primer PETE method” or “the FP-PETE method”). The use of two flanking primers is believed to increase the stringency of the enrichment step as compared to methods that require only a single primer or single bait for enrichment of each structurally distinct target polynucleotide. Thus, in some cases, the FP-PETE method provides improved or synergistic target enrichment in comparison to other target enrichment methods such as, e.g., single primer extension target enrichment methods.

Moreover, in contrast to multiplex PCR based methods in which multiple first and second amplification primers are in the same reaction mixture at the same time, the FP-PETE method can include a step of removing un-extended first primers before introducing second primers into a reaction mixture. Thus, in some cases, the method can reduce or eliminate competition between first and second primers. As such, in some cases, the first or second primers, or both can be used at significantly higher concentrations in the FP-PETE reaction mixture as compared to, e.g., multiplex PCR based methods. Additionally, or alternatively, an increased number of first or second primers can be used in the FP-PETE reaction mixture as compared to, e.g., multiplex PCR based methods.

It is believed that the use of (i) a large number of first or second primers, (ii) a high concentration of first or second primers, or (iii) a combination thereof, can provide improved enrichment for, e.g., high-throughput sequencing sample workflows in which a large number of different polynucleotide sequences are targeted and for which flanking hybridization sequences for the target sequences are known. Such high-throughput sequencing sample workflows include, but are not limited to, immune repertoire profiling workflows in which B cell receptor (BCR) or T cell receptor (TCR) sequences are enriched from a sample, sequenced, and analyzed. Flanking primer extension target enrichment methods for immune repertoire profiling workflows is termed “immuno-PETE” (see U.S. Pat. No. 11,098,360, the disclosure of which is hereby incorporated by reference herein in its entirety).

In another embodiment, the first or second primers are complementary or substantially complementary (i.e., at least 70%, 75%, 80%, 85%, 90%, 95% or 99% complementary) to a framework 1, framework 2, or framework 3 region of an immune cell receptor V gene. In another embodiment, the first or second primers are complementary or substantially complementary (i.e., at least 70%, 75%, 80%, 85%, 90%, 95% or 99% complementary across at least 5, at least 10, at least 15, at least 20 or more nucleotides) to a framework 1, framework 2, or framework 3 region of an immune cell receptor V gene. In another embodiment, the first or second primers are complementary across their full-length to a framework 1, framework 2, or framework 3 region of an immune cell receptor V gene.

In yet another embodiment, the first or second primers are complementary or substantially complementary (i.e., at least 70%, 75%, 80%, 85%, 90%, 95% or 99% complementary) to an immune cell receptor J gene region. In another embodiment, the first or second primers are complementary or substantially complementary (i.e., at least 70%, 75%, 80%, 85%, 90%, 95% or 99% complementary across at least 5, at least 10, at least 15, at least 20 or more nucleotides) to an immune cell receptor J gene region. In another embodiment, the first or second primers are complementary across their full-length to an immune cell receptor J gene region.

### Age-Related Disruptive T Cell Response Architectures in COVID-19

The most evident predisposing factor for COVID-19 severity is age, evoking the increased susceptibility of older persons to other newly emerged viruses, including West Nile and SARS1 (Peiris et al., 2003; Jean et al., 2007). However, although older persons are commonly cited as making poor vaccine responses (Goodwin, Viboud and Simonsen, 2006), they maintain considerable sequence diversity in their CD4+ and CD8+ T cell receptor (TCR) repertoires, albeit reduced relative to younger persons (Qi et al., 2014). It was therefore reasonable to ask whether the adaptive response architectures of COVID-19 patients displayed age-related traits. Calculating infection-to-fatality ratios in older groups is seriously challenged by variables such as heightened vulnerability of care-home residents (O'Driscoll et al., 2021). Nonetheless, some studies have reported a conspicuous inflexion in risk of fatality at age 50 (Docherty et al., 2020; Verity et al., 2020; Piroth et al., 2021), and in the United Kingdom, the Joint Committee on Vaccination and Immunization have recommended that individuals aged 50 or over should be first to receive COVID-19 vaccines in Phase 1 of the national vaccination program (Joint Committee on Vaccination and Immunisation: advice on priority groups for COVID-19 vaccination, 30 Dec. 2020—GOV.UK, 2020). In this study, patients and controls were parsed into those aged over and under 50 years, respectively.

To address these several issues, a newly developed, genomic (g)DNA-based technique permitting simultaneous characterization of antigen receptor repertoires for each of five lymphocyte subsets: CD4+ and CD8+ αβ T cells (TRB sequences); B cells (IGH sequences); and Vδ2+ and Vδ1+ T cells that respectively compose two distinct lineages of γδ T cells (TRD sequences) has been employed (see Examples 1-14, herein). Studying 95 individuals comprising hospital-treated COVID-19 patients and seropositive and seronegative controls, clear adaptive response patterns in each lymphocyte subset studied were clearly found, even in settings of profound immune dysregulation. However, whereas those response patterns were buffered by the scale and diversity of the systemic lymphocyte compartments in those aged under 50, TCR repertoire changes in those aged ≥50 commonly had global impacts that disrupted overall repertoire diversity. Such extreme focusing may not be beneficial, since recurrent IgVH sequences shared by many SARS-CoV-2-exposed individuals included Spike-specific, non-neutralizing antibodies that may be self-reactive.

### Results

### DNA-Based Repertoire Sequencing in COVID-19

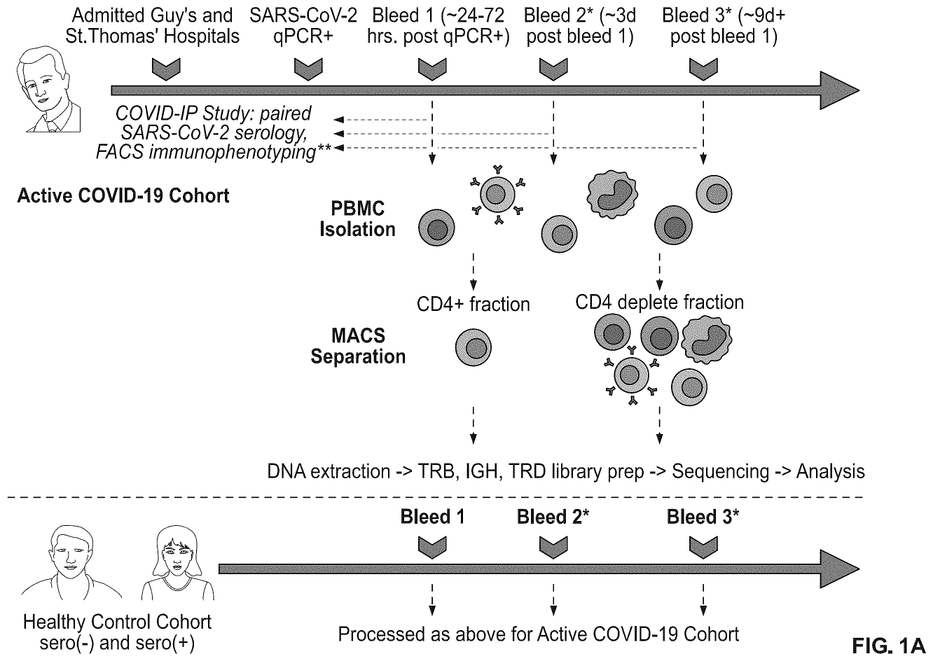

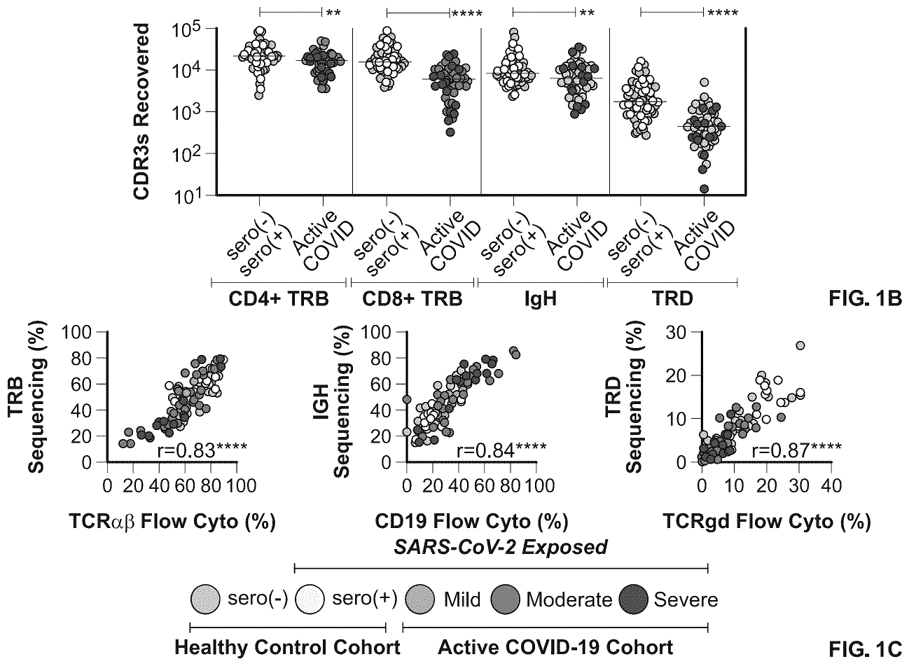

We sought to compare the Variable region repertoires for the immunoglobulin heavy chain (IGH) and TCRβ and TCRδ gene loci (TRB and TRD) of: 32 hospital-treated COVID-19 patients (active COVID-19) experiencing a range of disease severities; 20 healthy convalescent SARS-CoV-2-exposed individuals who had not required hospitalization [sero(+)]; and 43 non-exposed healthy adult controls [sero(−)]. These cohorts included several individuals aged ≥50 years (FIG. 7A), the inflection point at which the likelihood of COVID-19-associated death may increase greatly (Docherty et al., 2020; Verity et al., 2020; Piroth et al., 2021). To achieve this, the protocol described herein was applied to a subset of individuals who spanned a range of ages and disease severities, as described in the COVID-IP study (Laing et al., 2020), in which extensive immune profiling, serological analyses, and clinical annotation were undertaken (see FIG. 1A; note that the color-code for each cohort is maintained throughout all the results figures that follow). As expected, COVID-19 patients in the mild disease sub-cohort were enriched in females, whereas males were more common in the severe sub-cohort (FIG. 7B).

To assess the IGH, TRB, and TRD repertoires, a newly-developed DNA-based sequencing method termed Immuno-PETE was utilized (see U.S. Pat. No. 11,098,360, the disclosure of which is hereby incorporated by reference herein in its entirety). The Immuno-PETE assay includes primer mixes concurrently targeting all known human IgVH, TCRβ, and TCRδ V and J genes coupled with the use of unique molecular identifiers (UMIs). After sequencing, UMI and complementarity determining region 3 (CDR3) sequences were identified and jointly used to cluster reads originating from the same molecule. Consensus sequences were derived from read clusters, suppressing sequencing and PCR errors in the datasets. Through this use of UMIs, an accurate prediction of each CDR3 sequence was able to be derived. Additionally, since genomic (g)DNA was targeted, it was possible to achieve single molecule resolution, offering accurate counts of each cell type and further improving our measure of clonal diversity (see FIG. 7C).

Average sequence read-lengths ranged from 170-210 nucleotides (1st quartile-3rd quartile) thereby capturing comprehensive information on V, D, and J gene usage and the unique P-nucleotide-mediated and other template-independent insertions and deletions that contribute to the variable lengths and composition of each CDR3. All non-productive V-D-J rearrangements were excluded, as were any artifactual hybrid sequences, e.g. Vδ-Jβ (FIG. 7C). Moreover, the three gene segment repertoires chosen are those that have the greatest capacity to reflect B cells, αβ T cells and γδT cells, respectively, because they show the highest rates of allelic exclusion and expression fidelity. By comparison, productive Vγ gene segment rearrangements show only variable allelic exclusion in γδ T cells, and are common in αβ T cells (Sherwood et al., 2011). Finally, Vδ1 can occasionally be productively rearranged to J-Cα in αβT cells, but such cells were not detected because no Jα primer sequences were included; hence, the Vδ1 sequence counts reflected bona fide γδT cells.

To estimate the repertoires of five lymphocyte sub-types with bona fide adaptive potential, CD4+ T cells from freshly harvested PBMC were purified by magnetic bead separation (see Examples 3 and 5 herein). Since there are very few CD4+ γδT cells in human blood, this was considered to be de facto a source of CD4+αβT cells. The CD4− T cell fraction was similarly considered as a source of: CD8+ αβ T cells (since very few CD4−CD8− αβT cells exist in blood); B cells; and γδT cells that comprise two main lineages, Vδ2+ and Vδ1+. The effective application of Immuno-PETE was evident in our obtaining on average ˜23,000 productive TRB CDR3s from 500 ng of DNA derived from sorted CD4+ T cells (FIG. 1B). The slightly lower recovery of CD4+ TRB CDR3s from active COVID-19 patients may reflect T cell dysregulation that is an overt feature of COVID-19. Indeed, particularly severe cytopenia observed for CD8+ T cells and Vδ2+T cells in in COVID-IP, and in other studies (Carissimo et al., 2020; Diao et al., 2020; Laing et al., 2020; Rijkers, Vervenne and van der Pol, 2020), was reflected in those cells showing the poorest sequence recoveries from the CD4− fraction, albeit that the order of sequence recoveries (CD4−>B>>γδ) also reflected typical PBMC composition in most cases (FIG. 1B). Importantly, the consistent performance of Immuno-PETE was evident from the striking correlation of recovered IGH, TRB, and TRD CDR3 sequences with flow cytometric cell enumeration of parallel aliquots of the samples sequenced (Spearman r>0.8, p<0.0001) (FIG. 1C) (see Example 6 herein). Given the robust and quantitative nature of Immuno-PETE, equivalence of productively rearranged CDR3s was drawn to cell counts, a frame-of-reference employed from this point on. In total, 250 NGS libraries, one each for CD4+ and CD4− fractions, were constructed and analyzed. These included some longitudinal samples, primarily from COVID-19 patients. Our analysis provides data derived from ˜6.5 million antigen receptor chain sequences.

### Age-Related TCRβ Sequence Focusing in COVID-19

For each adaptive antigen receptor repertoire, the impact of COVID-19 and/or SARS-CoV-2 exposure on overall diversity was assessed by employing one or more of several entropic and dominance measures, including Shannon entropy (a.k.a. Renyi 1) and D50, which assign high values for diverse, entropic repertoires; and Simpson's dominance which assigns high values to more focused, clonal repertoires. Samples analyzed were split into “early” and “late” for those obtained up to and beyond 14 days post symptom-onset, respectively, and “unknown” for those who were asymptomatic sero(+) individuals in the community or asymptomatic hospitalized donors admitted for unrelated conditions. This temporal division is important if COVID-19+ patients aged ≥50 are to be compared with younger patients who invariably recovered and were discharged within two weeks.

As an example of this analysis, CD4+ T cells from early samples of SARS-CoV-2 exposed individuals aged ≥50 displayed a significantly lower Shannon entropy and higher Simpson's diversity relative to sero(−) controls aged ≥50 (FIG. 8A). For CD8+ T cells, entropy values were lower among controls compared to CD4+ T cells, consistent with the greater degree to which CD8+ T cells expand in response to myriad environmental exposures (Zhang and Bevan, 2011; Li et al., 2016). Nonetheless, there was a trend toward decreased Shannon's entropy for early samples of SARS-CoV-2 exposed individuals aged ≥50 compared to sero(−) controls aged ≥50, and a significant increase in Simpson's dominance (FIG. 8B). Note that although distinct entropy and dominance treatments are mostly related measures, they can show differential significance based, for example, on uneven influences of dominant clone sizes. By contrast, there were no significant differences in entropic or dominance measurements for SARS-CoV-2 exposed individuals aged under 50 relative to sero(−) counterparts (FIGS. 8A and 8B).

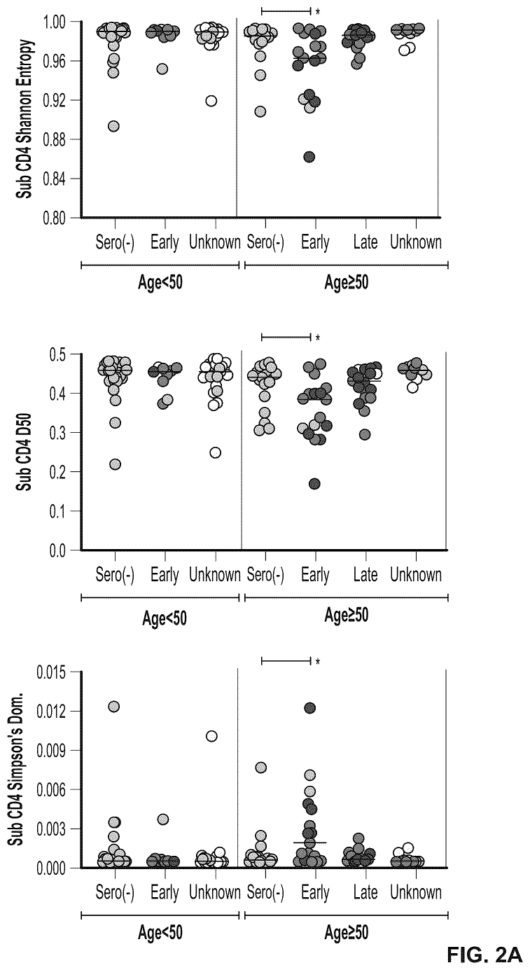

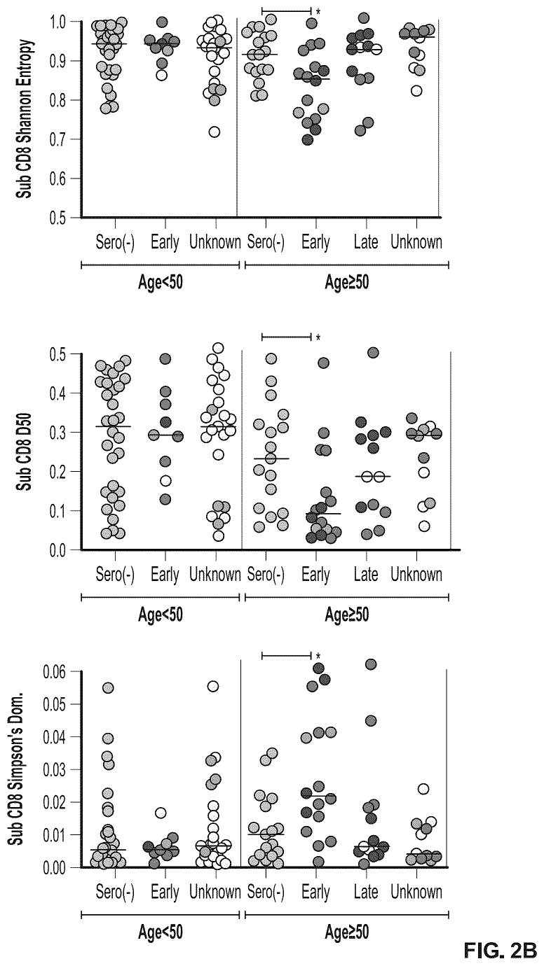

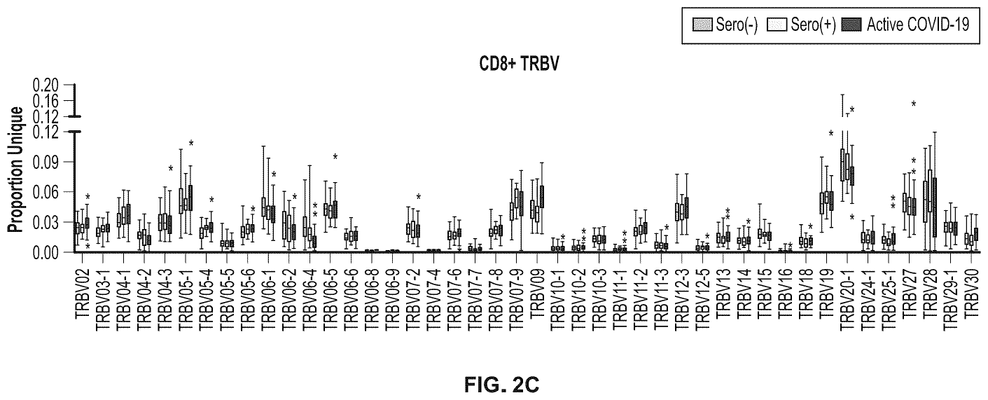

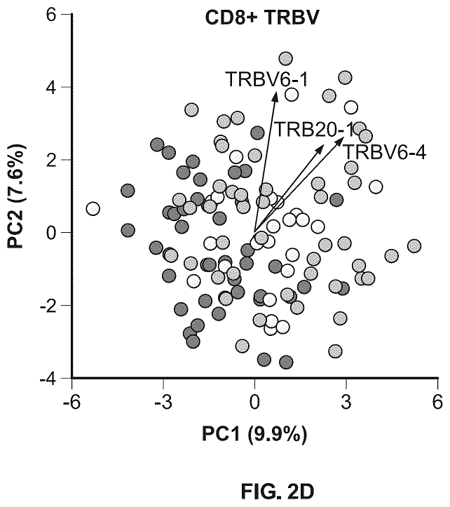

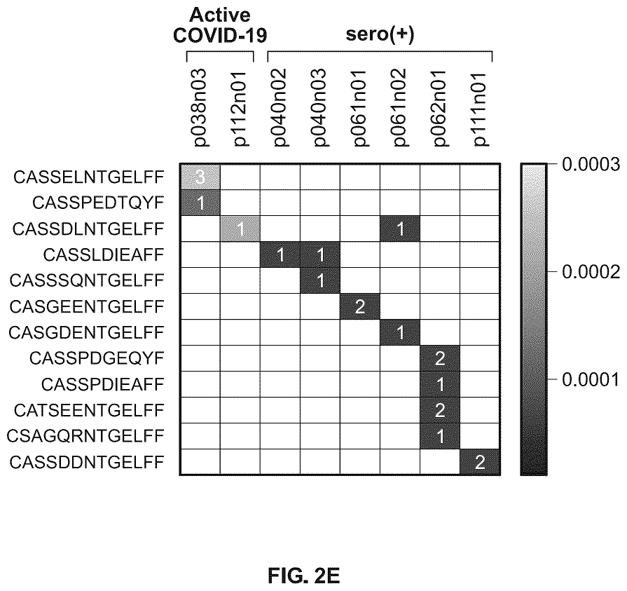

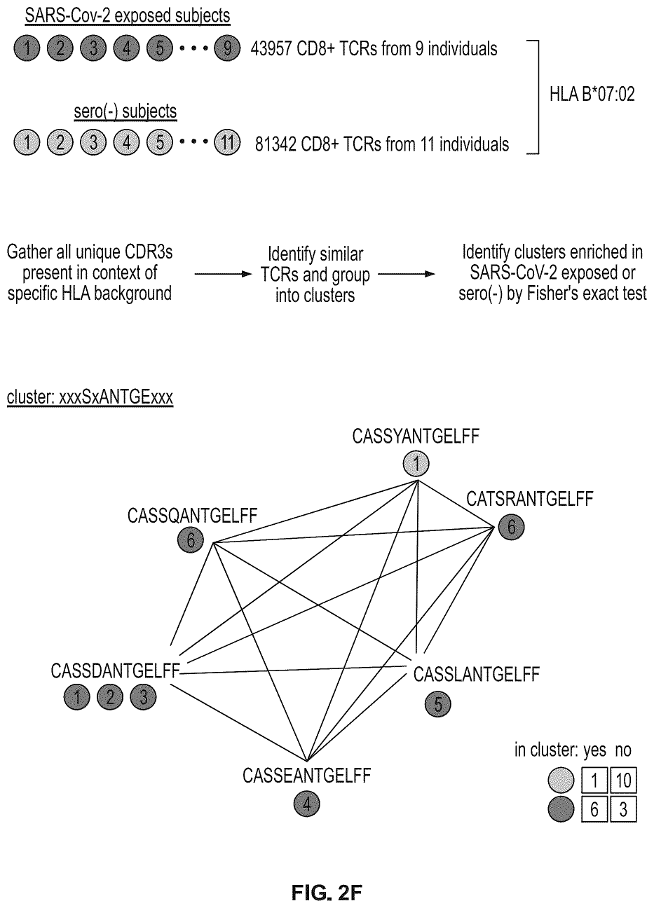

High diversity is disproportionately contributed to by unexpanded, antigen-inexperienced T cells that compose the bulk of CD45RA+CCR7+ αβ T cells, as was evidenced across all the cohorts in this study by the correlation of flow cytometry phenotyping and sequence data (FIG. 8C). Given that antigen-inexperienced cells are not those most relevant to SARS-CoV-2 responses, their influence can be minimized, by appropriate sub-sampling (see Methods). When this was undertaken for CD4+ T cells, Shannon's entropy was again significantly reduced for early samples of SARS-CoV-2 exposed individuals aged ≥50, with increased clonality also revealed by a lower D50 and higher Simpson's dominance in this cohort compared to age-matched sero(−) controls. (FIG. 2A). For sub-sampled CD8+ T cells, early SARS-CoV-2 exposed samples from individuals aged ≥50 were likewise significantly different from sero(−) samples as judged by Shannon entropy, D50 and Simpson's dominance (FIG. 2B). In sum, TRB sequence focusing in active COVID-19 patients aged ≥50 was sufficient to significantly reduce the entropy of total CD4+ and CD8+ TRB repertoires. Again, neither was true for those aged <50 (FIGS. 2A,B). Interestingly, by every entropy/dominance measure, the TRB repertoires of those aged ≥50 returned toward normal at later time-points post symptom-onset, suggesting the potential for relatively rapid renormalization. Additionally, it is evident from the color-coded data (FIGS. 2A,B) that those with the highest degree of focusing included patients experiencing a spectrum of disease severities, arguing against the possibility that it is primarily a consequence of pathology.

One means of reducing diversity is for SARS-CoV-2 to encode and/or induce a superantigen(s) (SAg) that elicits responses from T cells expressing defined Vβ regions. This has been speculated to occur in COVID-19, and some evidence for it has been provided for post-SARS-CoV-2-associated, pediatric MIS-C (Cheng et al., 2020; Porritt et al., 2021). Our analyses of repertoires (appropriately subsampled to correct for variable CD4+ and CD8+ T cells recovered) showed that there was no disproportionate enrichment or gross depletion that would be most easily explained by Vβ-specific SAgs for either CD4+ (FIG. 8D) or CD8+ T cells (FIG. 2C). However, there was a relative depletion among active COVID-19 CD8+ T cell repertoires of TRBV6-1, TRBV6-4, and TRBV20-1, that collectively could segregate active COVID-19 patients from sero(+) or sero(−) subjects by principal component analysis (PCA) (FIG. 2D). These Vβ regions are disproportionately utilized by MAIT cells which have been reported to be selectively depleted in COVID-19 patients (Jouan et al., 2020; Flament et al., 2021).

### TCRβ Sequence Sharing and Clustering in COVID-19

A more conventional means by which TCR diversity could be reduced would be via expansions of TCR sequences specific for SARS-CoV-2 peptides. In this regard, of 344 TCRs recently reported to bind SARS-CoV-2 Spike (S) peptide, YLQPRTFLL presented by HLA-A*0201 (Shomuradova et al., 2020), an overlap of 12 TRB sequences was identified, solely among HLA-A*0201+ active COVID-19 samples (n=2) and sero(+) (n=6) samples, with the latter including two pairs of longitudinal samples (FIG. 2E; p040n02, p040n03; p061n01, p061n02) (HLA Typing is described in Example 8, herein). Although this degree of overlap might seem slight, it was greater than the overlap reported when two different assays (tetramer-staining versus cytokine-release assays) were used to identify YLQPRTFLL-reactive T cells in the primary source study (Shomuradova et al., 2020). Both observations suggest that shared sequences are relatively rare, but nevertheless the overlap was striking given that it occurred in 6 of 17 SARS-CoV-2 exposed individuals who were HLA-A*0201+, and none of 21 HLA-A*0201+sero(−) individuals. Moreover, one overlapping sequence was shared by one active-COVID-19 patient and one sero(+) individual, and given the reduced, cytopenia-associated sequence depth that is common in active-COVID-19, it was not surprising that this sequence, as well as other putative YLQPRTFLL-reactive sequences, accounted for a larger fraction of the TRB repertoire in active COVID-19 than was the case in recovered sero(+) individuals (FIG. 2E; color-scale). Additionally, different YLQPRTFLL-reactive sequences emerged over time in the longitudinal samples (FIG. 2E).

Given the great diversity of SARS-CoV-2 peptides to which an exposed individual's TCRs might react, and given that identical reactivities are shown by related but distinct TCR sequences, adaptive T cell responses may be assessed by tracking clusters of related TRB sequences. To this end, DNA typing was used to identify discrete MHC Class I (n=21) and Class II alleles (n=28) shared across at least 8 individuals, and in each case, the unique CDR3s were sampled; in total, >2 million unique TRB sequences from CD4+ T cells, and >800,000 TRB sequences from CD8+ T cells (Table 1 below which lists the number of unique TCRs available for clustering, number of clustered TCRs and significantly enriched clusters in CD4+ and CD8+ compartments. Significance established by one-sided Fisher's exact test p<0.05.). As an example, 43,957 CD8+ TRB sequences were contributed by 9 HLA-B*07:02+ SARS-CoV-2-exposed individuals and 81,342 CD8+ TRB sequences were contributed by 11 HLA-B*07:02+sero(−) individuals (FIG. 2F). These sequences were grouped with GLIPH2 (Huang et al., 2020) into clusters based on the inference from related amino acid sequences of structural and biochemical properties that predict similar peptide-MHC specificities (see Methods). In the illustrative HLA-B*07:02 cluster shown (FIG. 2F), sequences were contributed by 6 SARS-CoV-2 exposed individuals and by 1 sero(−) individual. Hence, the cluster is enriched in TCRs from SARS-CoV-2 exposed individuals (Fisher's exact, p<0.05). When assessed using Fisher's exact test, 2,993 clusters, containing 21,869 TCRs, were over-represented in CD4+ T cells from SARS-CoV-2 exposed individuals, while 511 clusters containing 3,458 TCRs were over-represented in CD8+ T cells from SARS-CoV-2 exposed individuals. While other clusters were comparably represented among all cohorts, there was only one single case of a cluster significantly over-represented in sero(−) individuals in the CD4+ compartment (see Table 1). Hence, TRB clustering identified a pattern of SARS-CoV-2-associated adaptive T cell responsiveness against a backdrop of diverse TRB repertoires.

TABLE 1

| | | | | # TCRs in enriched | |
|---|---|---|---|---|---|
| | # unique | | # enriched clusters | clusters in SARS- | # enriched |
| | TCRs for | # of raw | in SARS-CoV-2 | CoV-2 exposed | clusters in |
| | clustering | clusters | exposed individuals | individuals | sero(−) |
| CD4+ | 2,091,619 | 11,269,019 | 2,993 | 21,869 | 1 |
| CD4− | 897,211 | 1,122,161 | 511 | 3,458 | 0 |

The identification of clusters enriched among SARS-CoV-2-exposed individuals would be consistent with focusing driven by viral and/or COVID-19-associated antigens. Indeed, TCRs in SARS-CoV-2-enriched clusters had a significantly higher convergence level, i.e. the number of nucleotide permutations coding for one amino acid sequence, than was evident for the global CD4+ and CD8+ cell repertoires (FIG. 8E), which is consistent with focusing being driven by protein function. Also consistent with clustering being driven by viral and/or COVID-19-associated antigens, the numbers of unique CD4+ TRB sequences contributed by COVID-19/sero(+) individuals to clusters, when normalized to each individual's repertoire size, were significantly greater than the numbers of unique sequences contributed to those same clusters by sero(−) individuals (FIG. 8F).

In sum, COVID-19-associated αβ T cell focusing was evidenced by multiple criteria, including SARS-CoV-2-exposed enriched clusters with high convergence, and TRB sequences shared with documented Spike-peptide reactive TCRs. However, whereas such focusing was well buffered in persons under 50, it significantly reduced the global diversity (entropy) of CD4+ and CD8+ TRB repertoires in those aged ≥50, implying that focusing was more disruptive in relation to TRB sequences available. Indeed, those subjects with the greatest degree of CD4+ TRB repertoire focusing were those with the greatest degree of CD8+ TRB repertoire focusing.

### Age-Related Adaptive Vδ1 Cell Responses

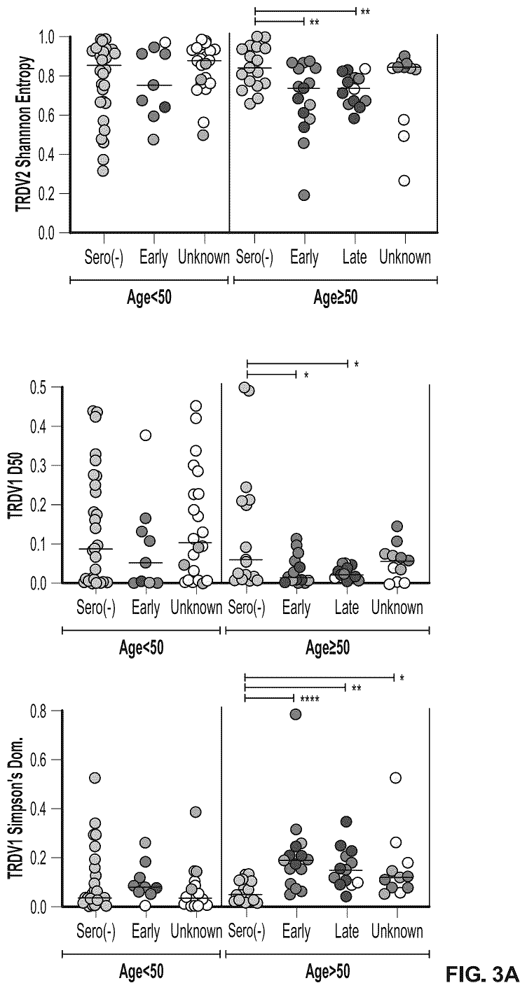

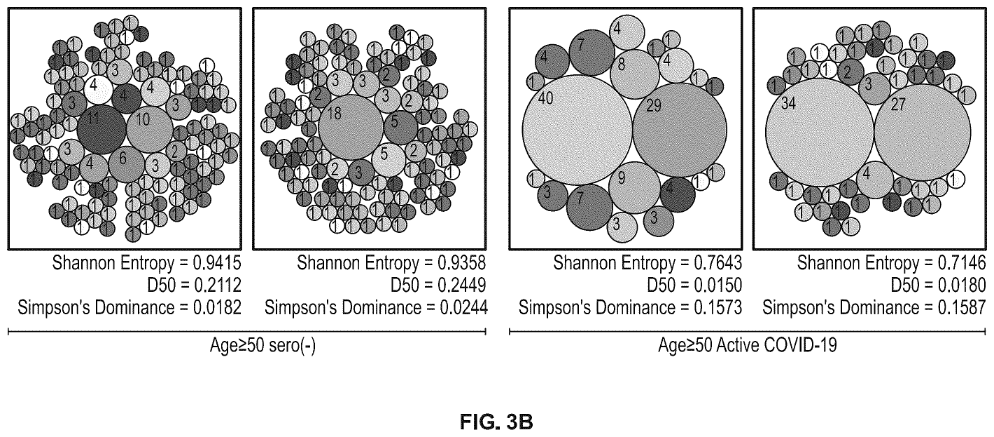

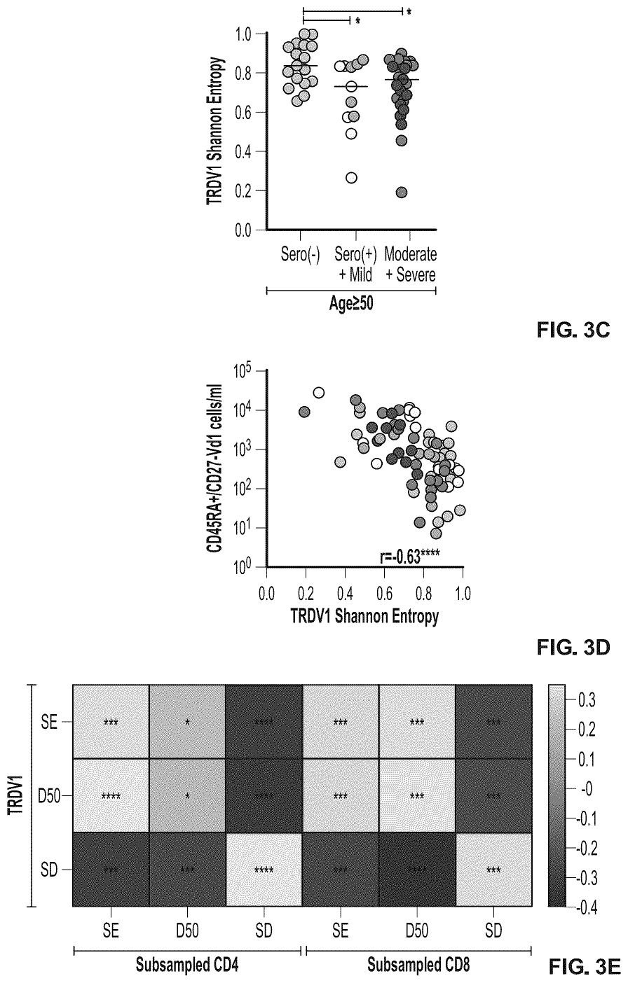

γδ T cells, that mostly comprise Vδ1+ and Vδ2+ T cells, compose a second, highly conserved T cell lineage with the potential to make adaptive responses to SARS-CoV-2 and/or COVID-19 (Hayday et al., 1985; Poccia et al., 2006; Laing et al., 2020; Rijkers, Vervenne and van der Pol, 2020). Indeed, in the COVID-IP study, increases in activated CD45RA+CD27− Vδ1+ T cells were one of only two immunological parameters to correlate with semi-quantitative measures of virus recovered from clinical swabs, the other being increased NK cell counts (Laing et al., 2020). Investigating the Vδ1+ T cell response, a significant reduction in Shannon entropy and D50 was observed as well as an increase in Simpson's dominance for Vδ1 (TRDV1) sequences in SARS-CoV-2-exposed individuals aged ≥50 compared to age-matched sero(−) controls (FIGS. 3A, 3B). This focusing in those aged ≥50 could be graphically illustrated by tree maps showing major expansions of a small number of clones in those exposed to SARS-CoV-2 (FIG. 3B).

Focusing was detected in early samples; was sustained in late samples; and was by some measures (Simpson's dominance) evident among sero(+) samples as well (FIG. 3A). Moreover, this age-related Vδ1 clonal focusing was seen in SARS-CoV-2 exposed patients regardless of disease severity (FIG. 3C), arguing that it was most likely driven by virus exposure, as considered above. Indeed, the decrease in TRDV1 Shannon entropy was significantly and strongly correlated with the expansion of CD45RA+CD27− Vδ1+ T cells (FIG. 3D) which were previously demonstrated to correlate with virus titres (Laing et al., 2020). Clonal expansions of these cells have been observed in the blood in different settings, including but not limited to CMV reactivation (Déchanet et al., 1999; Davey et al., 2017; Ravens et al., 2017; Rutishauser et al., 2020). By contrast, and as was true for TRB repertoires, there was no significant loss of entropy in cohorts of individuals aged <50 (FIGS. 3A, B).

There was a statistically significant correlation of Vδ1 focusing with TRB focusing (FIG. 3E), but in contrast to TRB (above) or IGH sequences (below), few TRDV1 sequences were shared across donors and none was significantly enriched in SARS-CoV-2 exposed individuals, suggesting that the Vδ1 responses in COVID-19 are primarily private. Likewise, very few public sequences were reported in other settings of Vδ1 focusing (Davey et al., 2017). Possibly, in each setting, Vδ1+ TCRs are not specific for pathogen-derived antigens but are specific for myriad molecular sentinels of dysregulation directly resulting from virus infection. Indeed, the CDR3 lengths which can be highly variable for TRD showed comparable distributions across SARS-CoV-2 exposed individuals, sero(−) individuals, and a prior analysis of human breast-resident Vδ1 TCRs (FIG. 9).

### Age-Related Vδ2 Cell Losses

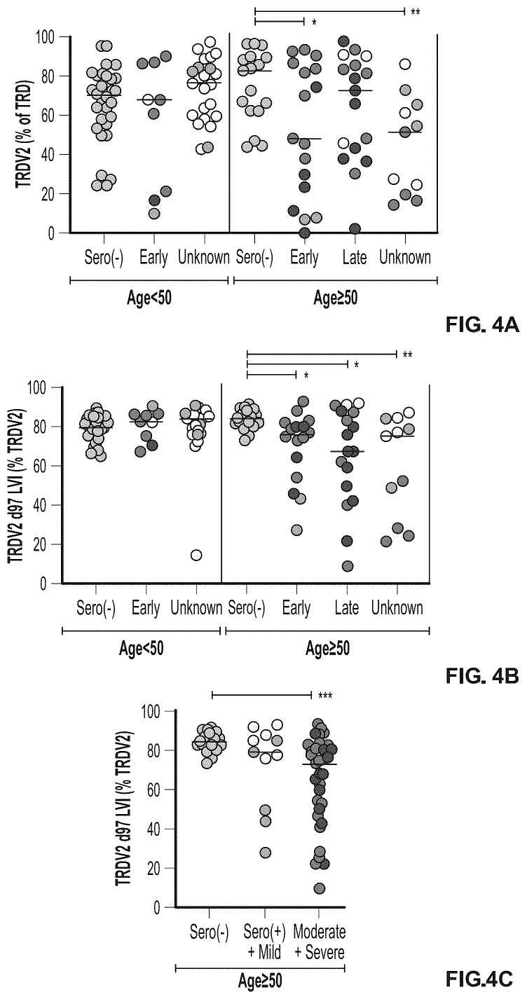

Ordinarily, the predominant blood γδ cell population comprises Vγ9Vδ2+ cells. Consistent with this, the majority of TRD reads were accounted for by Vδ2 (TRDV2) rearrangements in sero(−) individuals of all ages tested, and in SARS-CoV-2 exposed individuals aged <50, albeit that there was inter-individual variation as is well established for peripheral blood Vγ9Vδ2+ cells (Esin et al., 1996; Fonseca et al., 2020) (FIG. 4A). By contrast, the contributions of Vδ2+ cells were significantly reduced in SARS-CoV-2 exposed individuals aged ≥50 (FIG. 4A). Indeed, consistent with flow cytometry data from the COVID-IP study, >50% of early samples of COVID-19 patients aged ≥50 showed TRDV2 sequences collectively accounting for <50% of all TRD sequences (FIG. 4A). Moreover, this depletion from the blood was highly selective.

Most blood Vδ2-expressing cells are reactive to so-called phospho-antigens (PAgs) which are low molecular mass metabolic intermediates, including hydroxy-methyl-but-2-enol pyrophosphate (HMBPP) that is over-expressed by many bacteria and parasites, and isopentenyl pyrophosphate (IPP) that is over-expressed by many virus-infected cells, including those infected with influenza (Jameson et al., 2010). Vδ2 clonal focusing reactivity requires the pairing of Vγ9 with Vδ2 cells and a CDR36 that includes at position 97 a leucine, valine, or isoleucine residue (“d97 LVI”) (Yamashita et al., 2003). d97 LVI sequences account for ˜80% of the TCRVδ2 sequences in sero(−) individuals, but this was strikingly reduced to between 9% and 70% for many SARS-CoV-2-exposed individuals aged ≥50 (FIG. 4B). No such changes were evident in those aged <50 (FIG. 4B). Thus, there was a highly selective, age-related depletion of blood PAg-reactive Vδ2+ T cells. By contrast to Vδ1 TCR focusing, age-related d97 LVI depletion correlated with disease-severity although it was also observed in several individuals sampled who had experienced mild/moderate disease (FIG. 4C). Unlike, Vδ1+ T cells, there was no consistent evidence of clonal focusing in the Vδ2+ T cell compartment in relation to SARS-CoV-2 exposure, possibly manifesting its frequent classification as an innate-like compartment that responds en masse to PAg (Tyler et al., 2015; Hayday, 2019) (FIG. 10).

In sum, there were overt, disruptive changes in the γδ T cell compartment in SARS-CoV-2 exposed individuals aged ≥350, that were not evident in those aged <50. Those changes included Vδ1-focusing that was observed comparably in asymptomatic sero(+) individuals and in those experiencing severe disease, and which correlated with Vδ1 cell expansion; i.e. a bona fide age-related adaptive response. Conversely, age-related, selective Vδ2+ cell depletion was severity-related.

### IGH Sequence Focusing in COVID-19

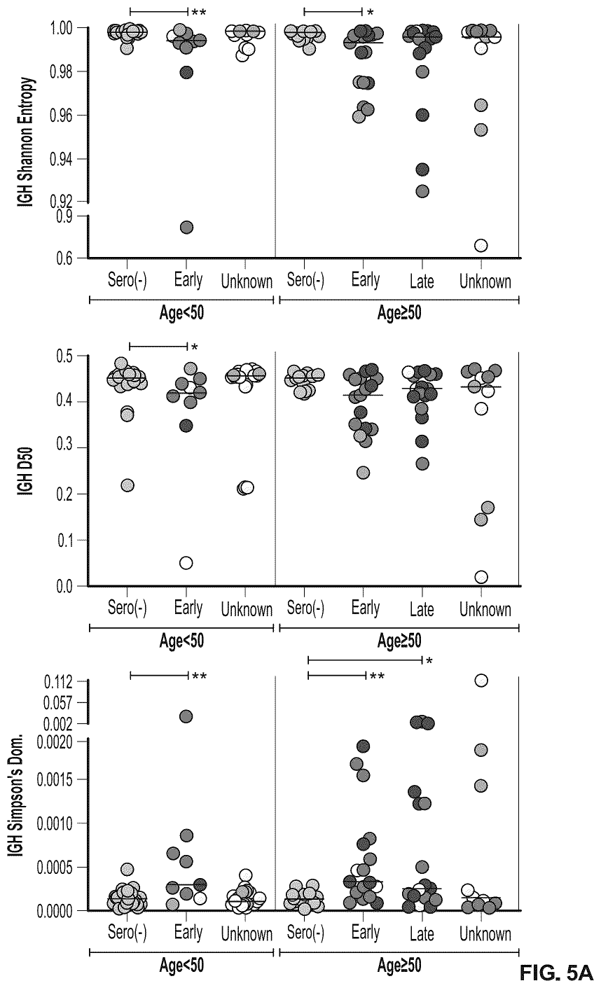

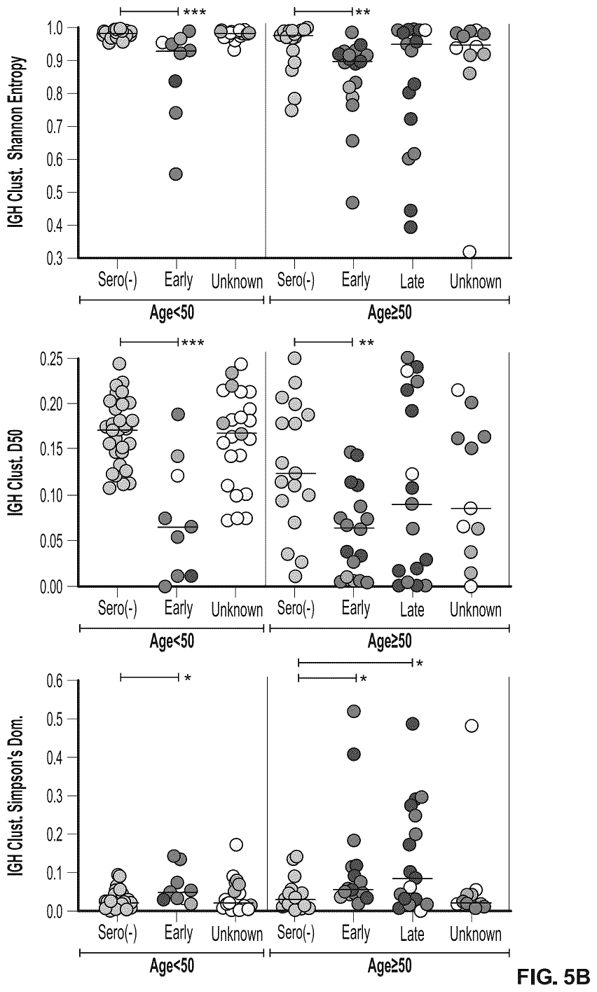

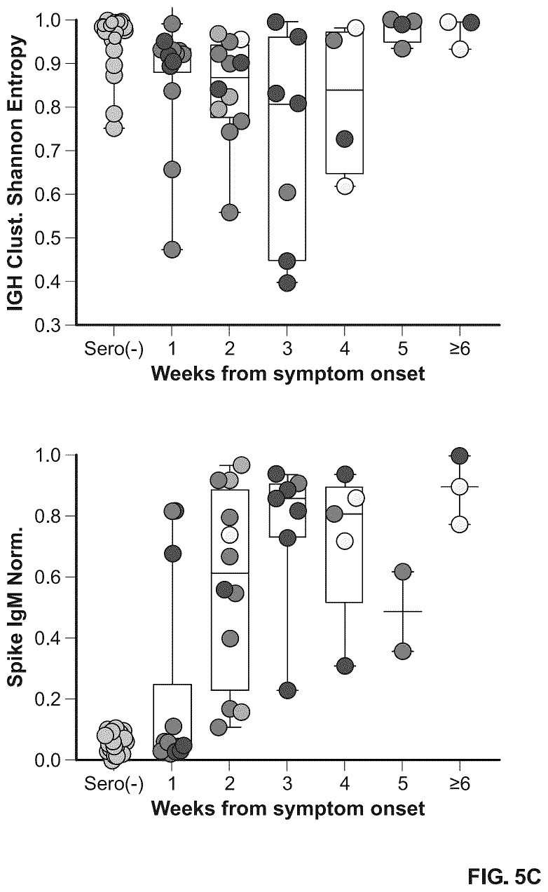

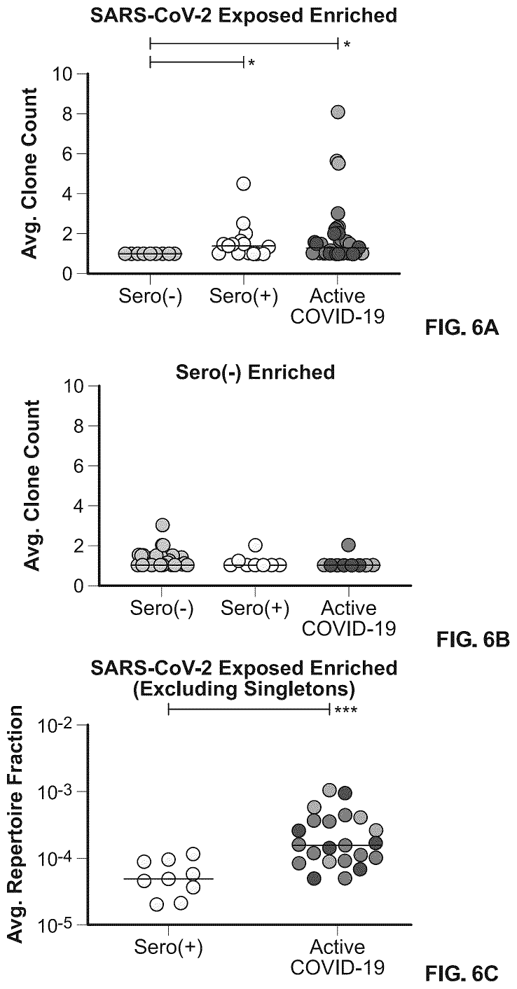

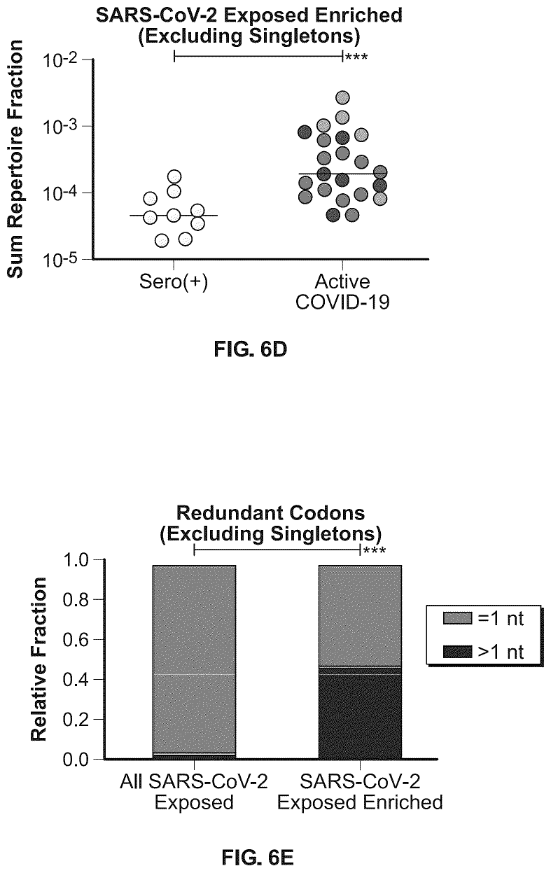

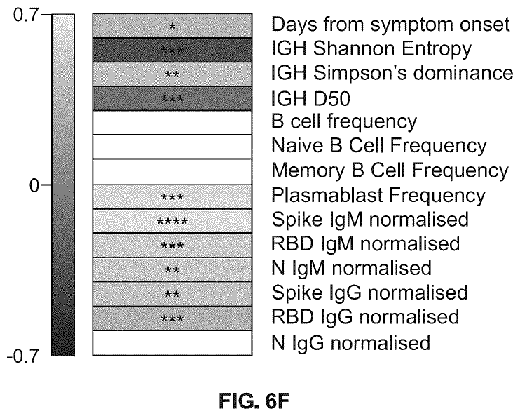

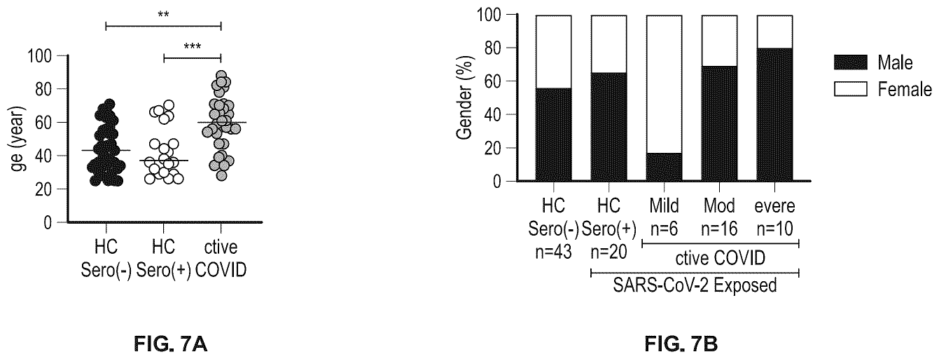

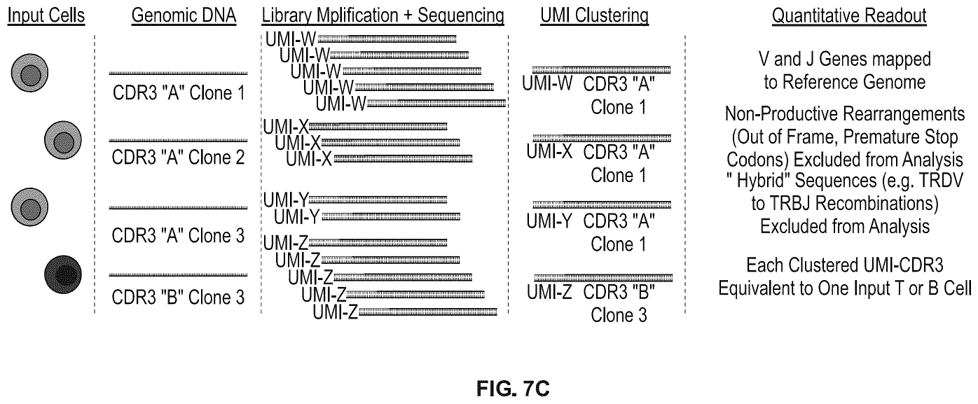

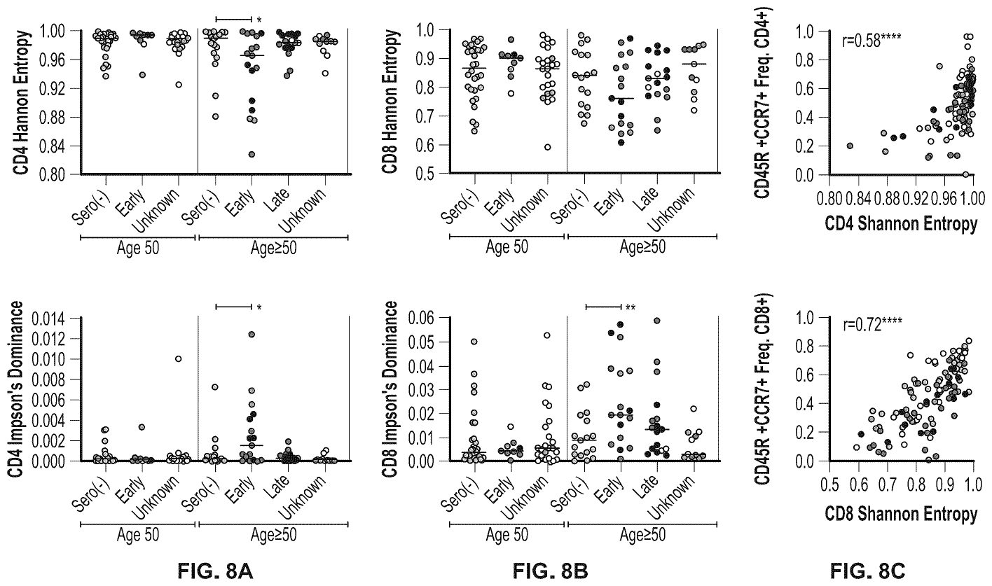

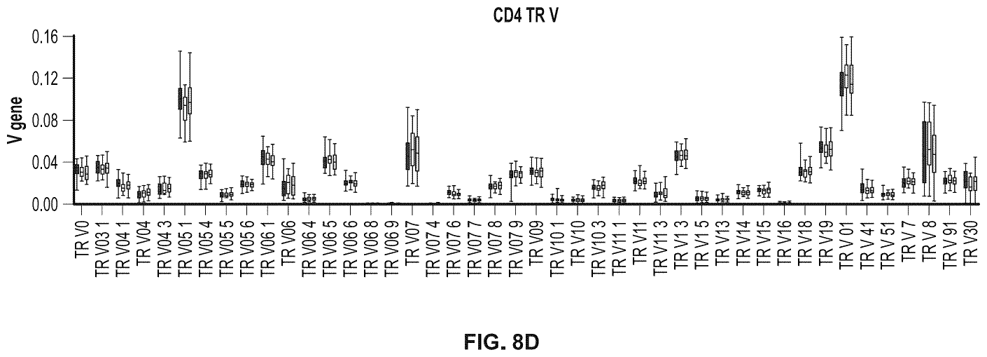

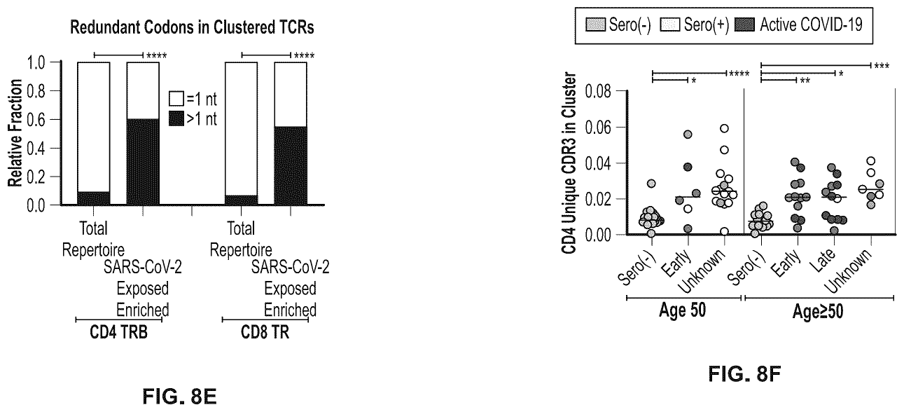

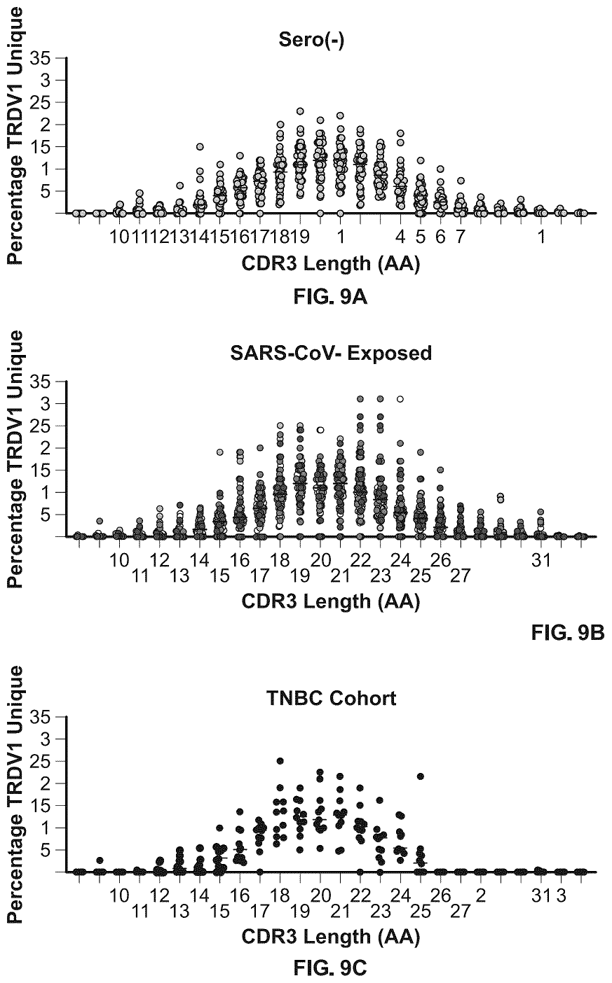

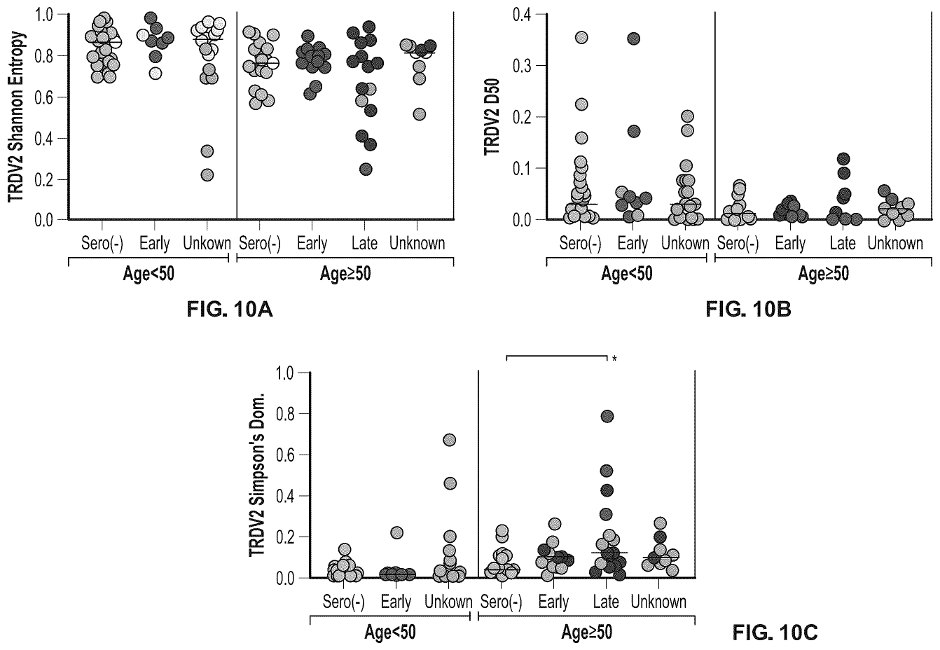

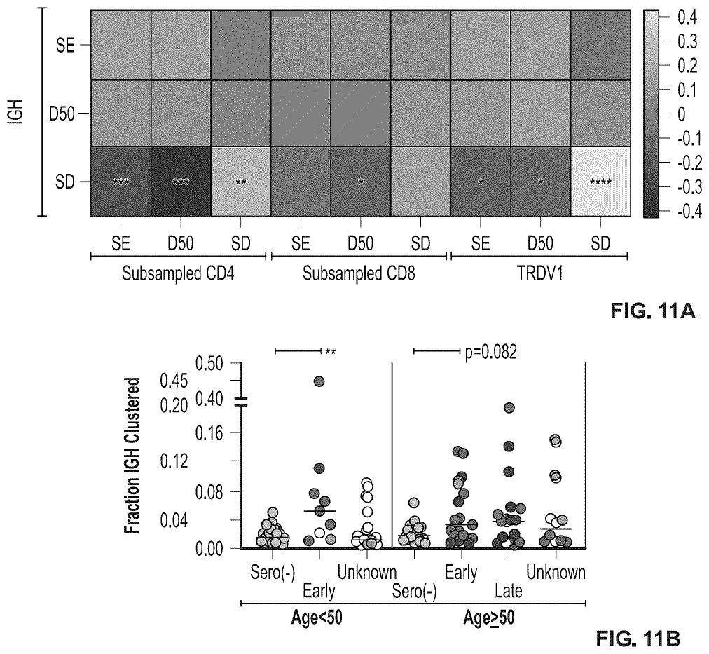

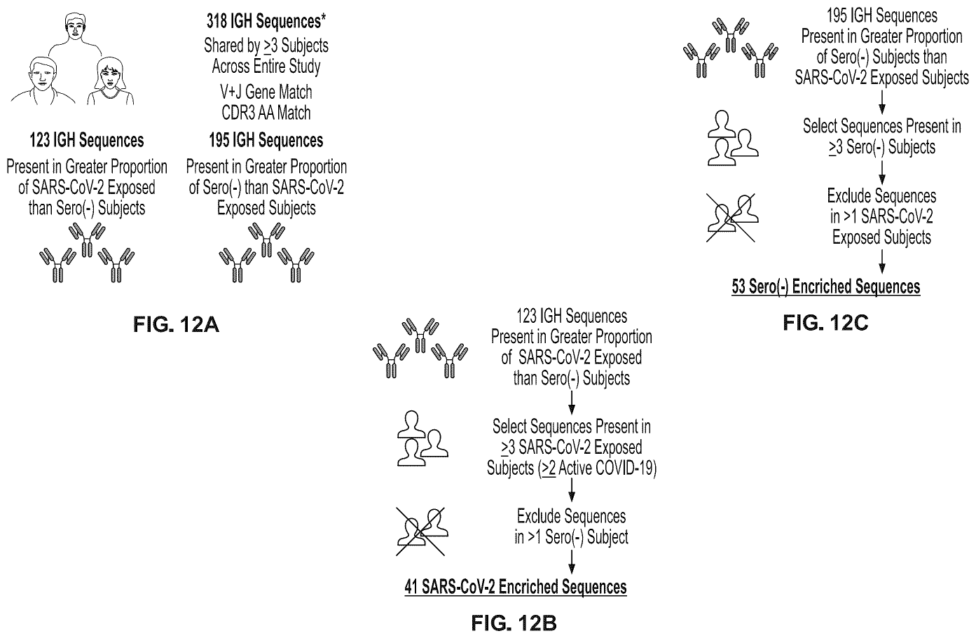

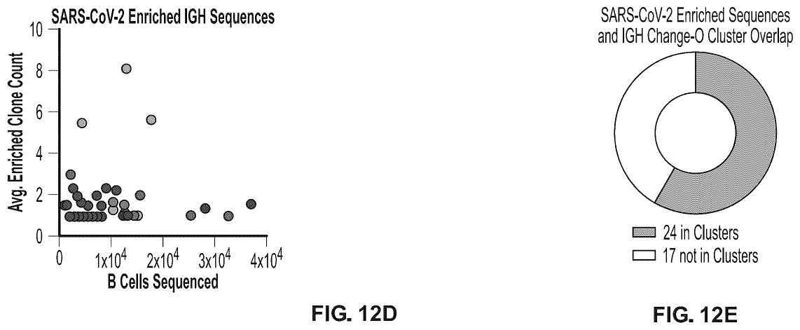

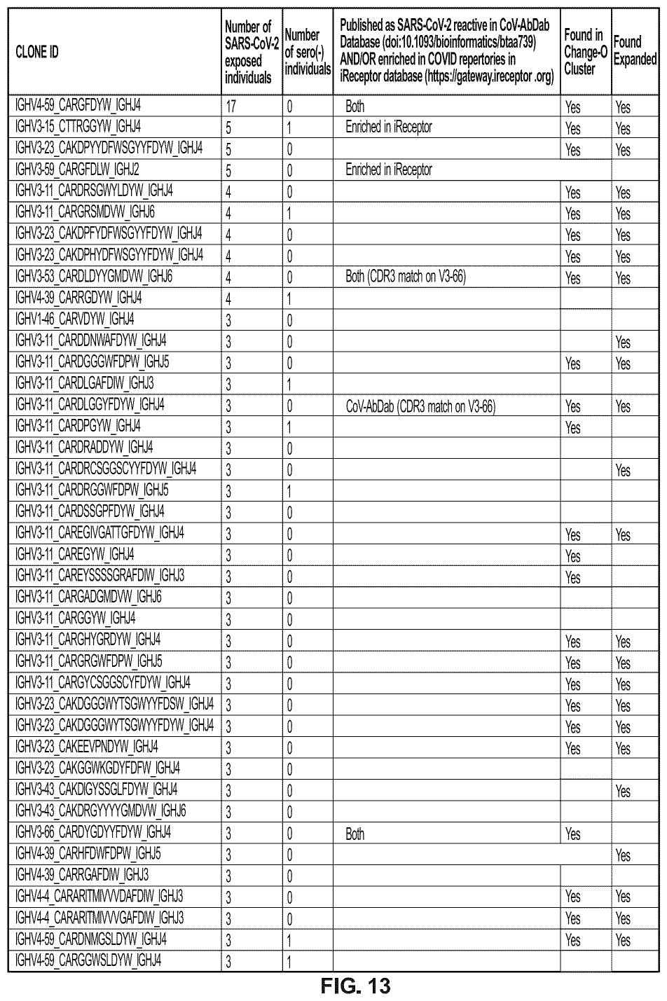

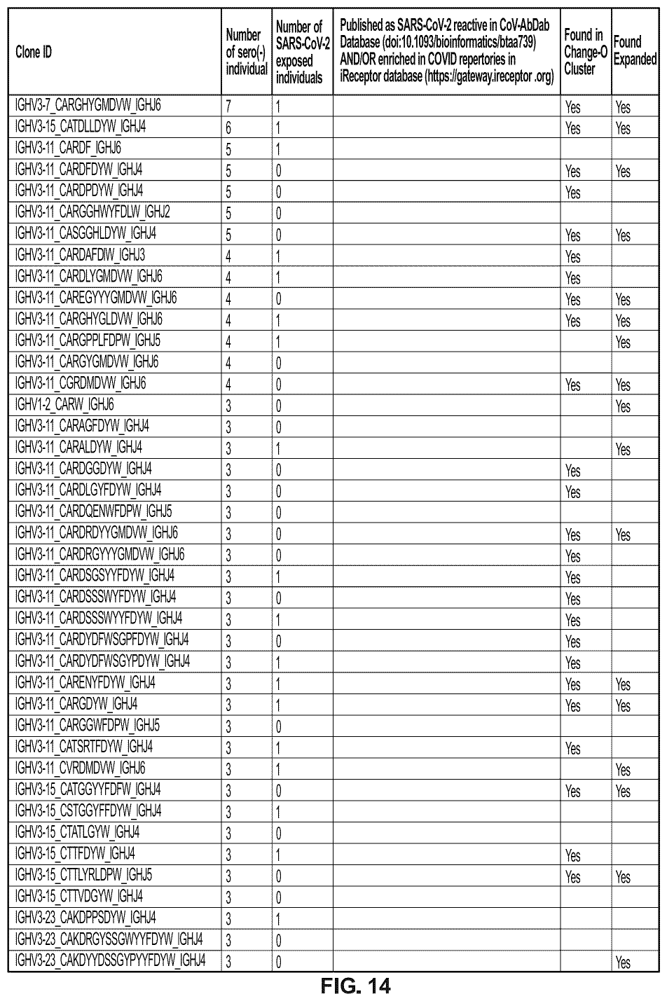

The COVID-19 IGH response showed a different pattern to the T cell response, with no overt age-related impacts, but with a cautionary revelation concerning the consequences of disruptive repertoire focusing. First, and by contrast to TRB and TRD, significant IGH focusing was evident in total sequence analyses for individuals aged <50 as well as those aged ≥50, when sampled within 14 days of symptom onset (FIG. 5A). Note that although those <50 included an outlier with remarkably overt focusing, there was no criterion by which to exclude that person, added to which there was a clear drop in Shannon's entropy for the bulk of the patients relative to controls and age-matched sero(+)individuals (FIG. 5A, upper left). Given that there was overt IGH focusing across age-groups, it was not surprising that it showed little correlation with age-dependent CD4+ TRB, CD8+ TRB, and Vδ1+ TRD focusing that were age-related (FIG. 11). As for TRB, however, the strongest IgVH sequence focusing was displayed by patients with a spectrum of pathologies, and largely renormalized at later time-points, albeit some legacy of focusing was revealed by Simpson's dominance (FIG. 5A).

The IGH landscape was next investigated in COVID-19 by asking the degree to which sequences within any single individual collectively composed clusters of related sequences (see Methods). This analysis revealed that relative to controls, there was an increase in the fraction of unique IGH sequences that could be found in clusters for early samples of SARS-CoV-2 exposed individuals aged <50 as well as those aged ≥50, although for the latter it was a trend rather than a significant difference (FIG. 11B). Moreover, the clusters in COVID-19 patients contained expanded clones because they had significantly lower entropy and higher dominance values than did clusters in control individuals (FIG. 5B). Confirming that those expansions were most likely driven by SARS-CoV-2/COVID-19, it was found that the temporal drop in Shannon entropy took place over the course of 3 weeks post-symptom onset, slightly preceding but largely overlapping with the development of Spike-specific IgM (FIG. 5C). In sum, COVID-19 was associated with overt IGH repertoire focusing, as individuals of all ages expanded clusters of related sequences coincident with the development of virus-specific antibodies.

### Shared IgVH Sequences Expanded in COVID-19

Independent evidence was next sought for SARS-CoV-2-reactive IGH sequences expanding in SARS-CoV-2 exposed individuals. Thus, 318 sequences were identified that were shared by at least three donors across the study (FIG. 12A). Of these, 41 were shared by at least 3 SARS-CoV-2 exposed individuals, of whom at least two were from the active COVID-19(+) cohort, and shared by no more than one sero(−) individual (FIG. 12B, FIG. 13): these were termed “SARS-CoV-2-enriched”. Conversely, 53 sequences were identified by a reciprocal approach as being enriched in at least 3 sero(−) individuals and shared by no more than one SARS-CoV-2 exposed individual; so-called “sero(−)-enriched” (FIG. 12C, 4). Strikingly, 6 of the 41 SARS-CoV-2-enriched sequences matched those reported by others to be SARS-CoV-2/COVID-19-associated (Raybould et al., 2020), or were significantly COVID-19 enriched in antigen receptor repositories (Corrie et al., 2018); over one half (24/41) were found expanded in at least one donor; and likewise over one half (23) were found in the clusters described above, thereby connecting these two independent manifestations of post-infection repertoire focusing (FIG. 6A, FIG. 13).

As anticipated, none of the 53 sequences sero(−)-enriched sequences were reported to be SARS-CoV-2/COVID-19-associated (4). Moreover, sero(−)-enriched sequences were not significantly expanded in those donors (FIG. 6B). Hence, the comparative expansions of SARS-CoV-2-enriched sequences in active COVID-19 individuals, as measured by average clone count per sample (FIG. 6A), were most probably driven by COVID-19-associated drivers. Certainly, they did not simply reflect the total B cells recovered because there was no correlation of their representation with IGH sequence recovery (FIG. 12D). Furthermore, by excluding singleton sequences from the analysis (in order to avoid any inappropriate clonal frequency bias arising because of cytopenia in some COVID-19 donors), it was found that the fractions of the IgVH repertoires composed on average or in sum by expanded SARS-CoV-2-enriched IGH sequences were significantly greater in active COVID-19 patients than in sero(+) individuals (FIGS. 6C,D). These sequences were never found to be expanded in sero(−) individuals. This amplification of shared sequences is consistent with the finding of reduced diversity in the sequence clusters of COVID-19 patients (above), and presumably reflects selection for particular IgVH specificities (Nielsen et al., 2020; Robbiani et al., 2020). Indeed, COVID-19 expanded sequences showed greatly increased convergence of nucleotide-to-protein sequence when compared to all sequences (singletons excluded) (FIG. 6E), analogous to the higher convergence of TCRVβ sequences seen in COVID-19/SARS-CoV-2-associated clusters (above).

Because over half the shared SAR-CoV-2-enriched clones were found in clusters of related sequences (FIG. 13; FIG. 6E), it was considered whether the occurrence of shared sequences and their close relatives correlated with other parameters of the COVID-19-associated immune response. Not surprisingly, they showed strong positive correlations with dominance metrics and strong negative correlations with entropy metrics applied to the global IGH repertoires as well as a correlation to time from symptom onset, consistent with an evolving adaptive response (FIG. 6F). Of note, however, they also showed strong positive correlations with plasmablast frequencies, with IgM specific for Spike, nucleoprotein (N) and Receptor Binding Domain (RBD), and with Spike and RBD-specific IgG. These findings validated the functional significance of the shared sequences and the sequence clusters as underpinning components of the B cell response to SARS-CoV-2/COVID-19. However, it did not necessarily equate to host benefit, since the most widely shared sequence CDR3-CARGFDYW- found in 17 SARS-CoV-2-exposed donors (FIG. 13) contributes to S-reactive but non-neutralizing antibodies previously identified in other studies (Cao et al., 2020; Raybould et al., 2020). Thus, focusing on the amplification of shared IgVH clones risks devoting a greater fraction of the repertoire to antibody clones of questionable utility.

### DISCUSSION

A newly-developed protocol for gDNA-based antigen receptor sequencing has been applied permits simultaneous characterization of IGH, TRB, and TRD repertoires for each sample. Being gDNA-based, it provided information about cell numbers that strongly correlated with flow-cytometry data and that may therefore prove powerful for samples, such as post-mortems, that are refractory to other forms of cell enumeration. Erroneous sequence calls were greatly reduced by including one or more UMIs, which is particularly important in relation to template-independent CDR3 sequences and somatic mutation in IGH. Aside from vaccination, and by contrast to cancer, there have been few opportunities to measure the establishment of adaptive immune responses to a known challenge incurred at a reasonably well defined time point. Moreover, there have been very few opportunities to measure the architectures of such responses in the context of overt immune dysregulation, sometimes in life-threatening settings, as evidenced for the same patients from whom antigen receptor sequences were obtained (Laing et al., 2020).

Given this, it is both striking and encouraging that strong and dynamic COVID-19-associated responses were observed in all three lymphocytic lineages with adaptive potential. Antigen receptor focusing in the B cell compartment was evidenced by four metrics: reduced entropy; increased dominance; greater clustering; and shared sequences. SARS-CoV-2 as a driver of these changes was inferred from the respective temporal dynamics of clustering and the development of SARS-CoV-2-specific serological responses, and from the identification of shared IgV-CDR3-IgJ sequences that are known to contribute to SARS-CoV-2-reactivity. Nonetheless, IGH sequence sharing was relatively rare, as it was for TRB even among MHC-matched individuals, suggesting that the bulk of SARS-CoV-2-reactive responses comprise private repertoires. Similar conclusions may be reached from other studies, notwithstanding some reports of prominent public IGH sequences (Galson et al., no date; Nielsen et al.; Schultheiß et al., 2020; Shomuradova et al., 2020).

The adaptive versus innate status of human γδT cells has been oft-debated (Davey et al., 2018; Willcox and Willcox, 2019; Hayday and Vantourout, 2020). While Vδ1+ clonal expansions have been reported in human blood and liver (Hunter et al., 2018), the provoking stimuli have not been clear with data for and against a role for cytomegalovirus (Davey et al., 2017; Ravens et al., 2017). In this context, our study provides an unique association of bona fide adaptive Vδ1 responses with a live virus challenge, as evidenced by the correlation of decreased entropy with Vδ1+ cell expansions, that were in turn correlated with viral-load. However, the lack of any COVID-19-associated sequence sharing supports prima facie the prospect that expanded γδ cell sequences may reflect reactivity to virus-induced changes in endogenous antigen displays rather than to the virus (Hayday, 2019).

Strikingly, and most unexpectedly, the Vδ1 responses had statistically significant impacts on entropy and dominance metrics of the whole blood Vδ1 repertoires only in persons aged >50. γδ T cell expansions seem commonly to occur in settings where αβ T cell responses are compromised, including immunosuppressed organ transplants (Déchanet et al., 1999), HIV-infection (Boullier et al., 2021), and endemic malaria. In COVID-19, cytopenia has been commonly reported, including in our cohort, particularly in CD8+ T cells, but this notwithstanding there were overt COVID-19-associated CD4+ and CD8+ T cell responses as judged by sequence clustering enriched in COVID-19/sero(+) individuals and sequence identities to known SARS-CoV-2-reactive TCRs. However, there was again a striking age-related impact, in that focusing in those <50 years did not impact the diversity (entropy) of the global TRB repertoire, whereas this was true for both CD4+ and CD8+ T cell compartments for many aged ≥50. In short, both the αβ and Vδ1 T cell responses to SARS-CoV-2/COVID-19 commonly had a disruptive impact on the TCR repertoires in those ≥50 years, begging two questions: the causes and the consequences.

In relation to cause, it is probably inappropriate to consider that those ≥50 are intrinsically T cell immunodeficient since they ordinarily harbor rich CD4+ and CD8+ T cell repertoires. However, those aged ≥50 may harbor greater percentages of senescence-associated CD57+, CD28−, p16+ T cells that are refractory to clonal expansion (Onyema et al., 2012), leaving responses to be dominated by larger expansions of smaller numbers of clones, as manifest in the down-sampled T cell entropy/dominance metrics for those ≥50 versus those <50. Moreover, an identical outcome may result from a need to respond to a newly-emerging pathogen via contributions from the naïve T cell compartment that in older persons features many distinct highly uneven expansions of private specificities (Qi et al., 2014).

Alternatively, an initial recruitment and expansion of pre-existing T cells primed to related antigens (e.g. seasonal coronaviruses) might also explain marked clonal focusing at early timepoints in individuals aged ≥50. Those individuals are more likely to harbor related memory T cells through accumulated exposure. However, whether or not those T cells are immunoprotective against SARS-CoV-2 remains unclear. This evokes similar findings demonstrating that early B cell responses after SARS-CoV-2 infection were enriched in cross-reactive memory B cells, including against seasonal coronaviruses of uncertain protective benefit, whilst late B cells responses were enriched in neutralizing SARS-CoV-2 RBD specific B cells (Sokal et al., 2021).

Clearly, less diverse repertoires of responding cells might collectively contain insufficient discrete reactivities to ensure virus neutralization and/or cytolysis of infected cells. Indeed, the most commonly-shared IGH CDR3 is documented as contributing to non-neutralizing SARS-CoV-2 S-reactive antibodies (Raybould et al., 2020). Likewise, with fewer clones available for expansion, it may take longer to compose an effective anti-viral compartment, which is of note given a recent association of disease severity with temporal delays in developing high-titre anti-SARS-CoV-2 antibodies (Lucas et al., 2020; Shen et al., 2020). Additionally, favored growth of pre-expanded “naïve” clones in older persons may also be problematic because of their reported self-reactivities (Qi et al., 2014). Because these outcomes would be probabilistic, the disruptive response architectures of those ≥50 would not necessitate a poor outcome; they would simply constitute a potentially significant added risk-factor, that might contribute to the upward inflexion point at 50 years for the probability of COVID-19-associated death. Note that our attempts to parse responses into other age groups failed to identify a clearer segregation of TCR responses than those between subjects over or under 50, respectively. Disruptive adaptive immunity might likewise have contributed risk to older persons encountering SARS1 and West Nile virus, when those infections first emerged in humans (Peiris et al., 2003; Jean et al., 2007).

It would be suggested that the potential contributions to disease may best be investigated in animal model systems where experimentally-engineered repertoire constraints could be assessed for their impacts on immunopathology induced by SARS-CoV-2 infection of human ACE2 transgenic mice. In the meantime, one TCR parameter emerged that was both severity-related and age-related; namely the loss of PAg-reactive Vδ2 sequences, and by extrapolation, PAg-reactive Vγ9Vδ2+ cells. Such cells are considered to be innate-like because of their high frequency among PBMC and hence their capacity to respond en masse. Conceivably, they are down-stream of severe pathology, for example, reflecting the cells' migration to the lungs in the context of severe respiratory disease. Alternatively, it seems striking that such depletions have been previously reported in settings, e.g. HIV infection, in which there is also overt but selective αβ T cytopenia, a signature of severe COVID-19 (Poccia et al., 1996; Li and Pauza, 2011). Such parallels serve as a reminder that age-related traits may concurrently impact several facets of lymphocyte biology, collectively jeopardizing an individual's responses to primary infection and/or associated co-infections.

### EXAMPLES

### Example 1—Human Subjects and Samples

Peripheral blood draws were obtained from patients and healthy control individuals as part of the COVID-IP study between 14 Apr. 2020 and 21 Jul. 2020 as previously described (Laing et al., 2020). The active COVID-19 cohort (n=32) included adult patients treated at Guy's and St Thomas' Hospitals (London, UK) with PCR proven SARS-CoV-2 infection on nasopharyngeal swab. Baseline peripheral blood samples in the active COVID-19 cohort were taken as soon as possible after a positive PCR result for SARS-CoV-2 (median=4 days, IQR=2-9.75 days). A subset of patients in the active COVID-19 cohort (13/32) had additional peripheral blood draws ˜3 days post baseline sampling and also variably at later timepoints. Patients in the active COVID-19 cohort were classified as having “mild” (not requiring supplemental oxygen), “moderate” (requiring less than 40% supplemental oxygen) or “severe” (requiring 40% supplemental oxygen and/or level 2 critical care) disease. Healthy control samples were obtained from 63 individuals drawn largely from a pool of healthcare workers and research scientists working at King's College London and Guy's and St. Thomas' Hospitals. A subset of these individuals (9/63) also had additional peripheral blood draws at later timepoints. All individuals had SARS-CoV-2 antibody titres determined by ELISA as previously described (Laing et al., 2020). Healthy control individuals were thus further categorized on the basis of SARS-CoV-2 serology as sero(+) (n=20) or sero(−) (n=43). A few sero(+) donors (3/20) were symptomatic and had previous PCR proven SARS-CoV-2 infection. The majority of sero(+) donors (17/20) had presumed asymptomatic infection without PCR evidence. A total of 125 peripheral blood samples were collected (including longitudinal timepoints) consisting of 52 active COVID-19 samples, 26 sero(+) samples and 47 sero(−) control samples.

Patient and healthy control samples were collected under the ethics approval of the Infectious Diseases Biobank of King's College London with reference numbers COV-250320 and MJ1-031218b respectively. Both approvals were granted under the terms of the Infectious Disease Biobank of King's College London ethics permission (reference 19/SC/0232) granted by the South Central Hampshire B Research Ethics Committee in 2019. We complied with all relevant ethical regulations.

### Example 2—PCR for SARS-CoV-2 Detection

Nasopharyngeal swabs were collected from patients suspected to have COVID-19 or for routine screening from those regularly attending or admitted to hospital for other reasons. Nucleic acid extraction and PCR were performed as previously described for the COVID-IP study using the AusDiagnostics two-step multiplexed-tandem PCR assay (Coronavirus Typing Eight-well Panel; cat. no. 2061901) or AusDiagnostics SARS-CoV-2, Influenza, RSV (eight-well) Panel (cat. no. 80081) (Laing et al., 2020).

### Example 3—Sample Processing and PBMC Isolation

Full methodology for sample processing is described in detail in the COVID-IP study (Laing et al., 2020). Briefly, whole blood samples were processed in Biosafety Level 3 containment conditions as per local code of practice approved by King's College London. Whole blood was diluted 1:1 with PBS and peripheral blood mononuclear cells (PBMCs) obtained by Ficoll density gradient separation. Approximately 20% of the PBMC fraction from each blood draw was used for downstream magnetic-activated cell sorting (MACS) and DNA extraction for antigen receptor sequencing and HLA typing as described below.

### Example 4—SARS-CoV-2 Serology

SARS-CoV-2 serology was determined by ELISA using diluted plasma from Ficoll density gradient separation as previously described in the COVID-IP study (Laing et al., 2020). Titers were normalized using a min/max normalization to compare samples across batches within the COVID-IP study. Cut-offs were determined based on data distribution with respect to healthy controls and values>0.15 were considered as positive. Sero(+) samples were positive for anti-spike and/or anti-RBD. Negative samples or samples positive only for anti-nucleoprotein were classified as sero(−).

### Example 5—MACS Sorting of PBMCs

PBMCs aliquots (˜2-20 million cells) from were MACS sorted with CD4 Microbeads (Miltenyi) yielding a highly pure CD4+ab T cell fraction and a CD4+ depleted PBMC fraction containing CD8+ab T cells, gd T cells and B cells. Sorting was carried out as per manufacturer's instructions with minor modifications to optimize the purity of both CD4+ and CD4− fractions. Briefly, PBMCs were counted, washed and resuspended in 80 ml of sterile MACS buffer (PBS, 2% fetal bovine serum, 2 mM EDTA) for every 5×106 cells up to a maximum of 320 ml for 2×107 cells. PBMCs in suspension were incubated with 20 ml of CD4 Microbeads for every 5×106 cells for 15 minutes at 4° C. then washed with MACS buffer before resuspending in 1 ml of MACS buffer. This suspension was then applied to an MS column (Miltenyi) in sequential 500 ml aliquots. Columns were washed three times with 500 ml of MACS buffer to collect the CD4− fraction. Columns were then forcibly flushed with 1 ml of MACS buffer to harvest the CD4+ fraction. Care was taken to keep reagents and cells at 4° C. to minimize activation and non-specific labelling. The cell suspensions for both positively and negatively selected fractions were washed, counted and resuspended in approximately 20 ml of MACS buffer. A small aliquot (˜2 ml) from each sample was taken for flow cytometry to assess relative frequencies of T and B cell subsets (see flow cytometry below). The remainder (˜18 ml) was lysed in RLT plus buffer+10 ml/ml of 2-mercaptoethanol (Qiagen) and frozen at −80° C. for subsequent nucleic acid extraction using the Allprep DNA/RNA mini kit (Qiagen, see DNA extraction below).

### Example 6—Flow Cytometry

MACS sorted cell fractions were stained for 15 mins at 4° C. in 50 ml of MACS buffer and antibody mastermix [anti-CD3 APC (Biolegend), anti-CD4 PerCP/Cy5.5 (Biolegend), anti-CD8 APC/Cy7 (Biolegend), anti-TCRgd PE/Cy7 (Beckman Coulter) and anti-CD19 FITC (Biolegend), all at 1:100 dilution]. Cells were then washed twice with MACS buffer and resuspended in fix buffer for 15 minutes (BD CellFIX). Fixed samples were acquired on a BD LSR Fortessa flow cytometer and results analyzed using FlowJo (Treestar/BD). CD3+ TCRgd-cells were considered to be ab T cells. Flow cytometry data presented of CD45RA+/CD27− Vd1+ T cell counts per ml of whole blood were generated as part of the COVID-IP study (Laing et al., 2020).

### Example 7—DNA Extraction, Quantification and Quality Assessment

DNA was extracted from cell lysates using the AllPrep DNA/RNA Mini Kit (Qiagen) as per manufacturer's instructions with minor modifications as detailed below. Briefly, lysates were homogenized using QIAshredder columns (Qiagen) as per manufacturer's instructions. Homogenized lysates were applied to AllPrep DNA spin columns and washed successively with Buffer AW1 and AW2 as per manufacturer's instructions. Fully washed columns were incubated at room temperature for 5 minutes with 50 ml of nuclease free water pre-heated to 70° C. and DNA eluted by centrifugation at ≥8000 g for one minute. The eluate was reapplied to the column for a second elution to maximize DNA yields. Quantification was performed using the Qubit dsDNA HS Assay kit (Thermo Fisher) as per manufacturer's instructions. DNA quality was measured by absorbance ratios at 260 nm/280 nm and 260 nm/230 nm using a Nanodrop spectrophotometer (Thermo Fisher).

### Example 8—HLA Typing

Approximately 500 ng of gDNA or 500 ml of whole blood from each donor was sent to Viapath Analytics (London, UK) for HLA typing using the Lifecodes Rapid SSO HLA Typing Kits.

### Example 9—Generating Antigen Receptor Libraries and NGS

A next generation sequencing (NGS) library of the adaptive immune repertoire of each sample was generated using the ImmunoPETE method. ImmunoPETE is a primer extension based targeted gene enrichment assay designed to specifically enrich and amplify human T-cell receptor (TCR) and B-cell receptor (BCR/Ig) loci from genomic DNA. It is optimized for the human TCRb (TRB), TCRd (TRD) and Ig heavy (IGH) chain receptors and uses Illumina NextSeq platforms for sequencing.

From gDNA, an initial single V gene-based primer extension was performed. V gene oligos contain a unique molecular identifier (UMI) sequence as well as a universal amplification sequence at the 3′ end. Following V gene-based primer extension, treatment with Thermolabile Exonuclease I (New England Biolabs) and a subsequent bead-based purification (KAPA HyperPure) removed remaining oligos. Thereafter, a master-mix of a pool of J gene oligos with an i7-primer was added to purified V gene-primed templates for J gene primer extension with a 10-cycle target amplification. This was followed by Illumina library amplification using i7/i5-sequencing primers with dual unique indexes. All primer extensions and amplifications were performed using the KAPA Long Range HotStart Ready Mix (Roche). Resulting libraries were purified using KAPA HyperPure beads (Roche), before quantification with the Qubit dsDNA HS Assay kit (Thermo Fisher) and fragment analysis on a TapeStation (Agilent). Libraries were pooled in equal mass to create a library pool before another round of quantification and fragment analysis before sequencing using the Illumina NextSeq 500/550 High Output Kit v2.5 (300 cycle).

### Example 10—Data Processing

A Roche in-house bioinformatics pipeline was used to process sequencing reads. ImmunoPETE leverages UMIs to enable counting of T and B cells at single molecule resolution. Read pairs were quality filtered and trimmed of adapter and primer sequences. V and J genes were identified by Smith Waterman alignment against the HGNC reference gene annotations. CDR3 regions were predicted for all V-J pairs, characterizing functional and non-functional rearrangements. UMI and CDR3 sequences were clustered together, defining UMI-families (all reads originating from a single molecule). In order to suppress errors that occur from sequencing or PCR, consensus sequences were derived for all UMI families with two or more reads. Quality score filters were also used to filter low-quality consensus reads, resulting in high quality CDR3 sequence predictions and accurate cell counts. Thus, ImmunoPETE provides unbiased and quantitative TCR and BCR repertoire information with next-generation sequencing analysis. Functional rearrangements were defined by V gene, CDR3 amino acid (AA) sequences, and J gene combinations excluding rearrangements containing pseudogenes. “Hybrid” rearrangements (e.g. TRBV-CDR3-IGHJ) were also excluded. All analyses were conducted using functional rearrangements (excluding hybrids) based on CDR3 AA sequences unless otherwise specified.

### Example 11—Diversity/Clonality Analyses

The frequencies of matching V genes+CDR3 AA+J genes were used to calculate entropy/dominance metrics across all 4 cell types (CD4+ ab T cells/CD4+ TRB, CD8+ ab T cells/CD8+ TRB, gd T cells/TRD and B cells/IGH). Metrics were normalized to account for differences in the total number of cells per sample and calculated as detailed in below.

$$R = number of clones ( each TCR may be present in several cells )$$

$$N = sum of cell counts across all clones = {\sum }_{i = 1}^{R} {n}_{i}$$

$$Frequency of each clone p : {\sum }_{i = 1}^{R} {p}_{i} = 1$$

$$Shannon ’ s entropy = {\sum }_{i = 1}^{R} {p}_{i} {log}_{2} ( {p}_{i} )$$

$$Simpson ’ s dominance = {\sum }_{i = 1}^{R} {p}_{i}^{2}$$

$${D}_{50} = \frac{C}{R} where clones are sorted decreasing and C is smallest possible C , where {\sum }_{i = 1}^{C} {n}_{i} \geq \frac{N}{2}$$

We used the DefineClones.py script in the change-o toolbox (version 1.0.0 2020.05.06) to cluster IGH CDR3 clones. Diversity measures were also calculated using IgH clusters, accounting for the impact of somatic hyper mutation (SHM) on the estimate of B-cell diversity.

### Example 13—TRBV Sub-Sampling

Each sample was sub-sampled to 1200 (CD8+) or 2400 (CD4+) cells by drawing TCRs (defined as V gene+CDR3 AA+J gene) with replacement with probability equal to their presence in the starting sample. Samples with less than 1200 (CD8+) or 2400 (CD4+) cells were not included. Medians of metrics computed from 100 resamples (entropy/dominance metrics, TRBV gene use frequency) were reported as “sub-sampled” values. TRBV gene usage was reported as a proportion of unique TCRs per sample.

### Example 14—TRBV Clustering

For each HLA gene, TCR sequences were collated from across all individuals from the study without sub-sampling (CD4+ for HLA class II and CD8+ for HLA class I), irrespective of their SARS-COV-2 exposure status. Only HLA backgrounds with at least 8 individuals and at least 4 SARS-COV-2 exposed individuals were included in subsequent analyses. Unique TCRs were clustered within each HLA background with GLIPH2 (http://50.255.35.37:8080/, executables from October 2020), ignoring the first and the last 3 amino acids of the CDR3 and with requirements for CDR3 length to be at least 8 amino acids, k-mers of size 2-4, allowing only BLOSUM62 positive amino acid replacements and with all other settings as default. Raw clusters were tested with a one-sided Fisher exact test for overrepresentation of SARS-CoV-2 exposed individuals (cut-off p<0.05). For the reciprocal comparison, the same analysis was performed, but conditioning on HLA presence in sero(−) individuals and requiring overrepresentation of this set of individuals in the clusters.

### Statistical Analysis

Statistical tests were conducted using Prism 9 (GraphPad) and R version 4.0.3+RStudio, with CRAN available packages. Statistical tests used are specified in accompanying figure legends. Where results of statistical test are shown; *p≤0.05, **p≤0.01, ***p≤0.001, ****p≤0.0001 unless otherwise indicated.

### REFERENCES

Le Bert, et al. (2020) ‘SARS-CoV-2-specific T cell immunity in cases of COVID-19 and SARS, and uninfected controls’, Nature, 584(7821), pp. 457-462. doi: 10.1038/s41586-020-2550-z.
Boullier, S., et al. (2021) HIV-infected persons. cell expansion in the peripheral blood of CDR3-independent gamma delta V delta 1+ T.
Cao, Y., et al. (2020) ‘Potent Neutralizing Antibodies against SARS-CoV-2 Identified by High-Throughput Single-Cell Sequencing of Convalescent Patients' B Cells’, Cell, 182(1), pp. 73-84.e16. doi: 10.1016/j.cell.2020.05.025.
Carissimo, G., et al. (2020) ‘Whole blood immunophenotyping uncovers immature neutrophil-to-VD2 T-cell ratio as an early marker for severe COVID-19’, Nature Communications, 11(1), p. 5243. doi: 10.1038/s41467-020-19080-6.
Cheng, M. H., et al. (2020) ‘Superantigenic character of an insert unique to SARS-CoV-2 spike supported by skewed TCR repertoire in patients with hyperinflammation’, Proceedings of the National Academy of Sciences of the United States of America, 117(41), pp. 25254-25262. doi: 10.1073/pnas.2010722117.
Corrie, B. D., et al. (2018) ‘iReceptor: A platform for querying and analyzing antibody/B-cell and T-cell receptor repertoire data across federated repositories’, Immunological Reviews, 284(1), pp. 24-41. doi: 10.1111/imr.12666.
Davey, M. S., et al. (2018) ‘Recasting Human Vδ1 Lymphocytes in an Adaptive Role’, Trends in Immunology, 39(6), pp. 446-459. doi: 10.1016/j.it.2018.03.003.
Davey, M. S., et al. (2017) ‘Clonal selection in the human Vδ1 T cell repertoire indicates γδ TCR-dependent adaptive immune surveillance’, Nature Communications, 8(1), p. 14760. doi: 10.1038/ncomms14760.
Déchanet, J., et al. (1999) ‘Implication of γδ T cells in the human immune response to cytomegalovirus’, Journal of Clinical Investigation, 103(10), pp. 1437-1449. doi: 10.1172/JCI5409.
Diao, B., et al. (2020) ‘Reduction and Functional Exhaustion of T Cells in Patients With Coronavirus Disease 2019 (COVID-19)’, Frontiers in Immunology, 11, p. 827. doi: 10.3389/fimmu.2020.00827.
Docherty, A. B., et al. (2020) ‘Features of 20 133 UK patients in hospital with covid-19 using the ISARIC WHO Clinical Characterization Protocol: Prospective observational cohort study’, The BMJ, 369. doi: 10.1136/bmj.m1985.
Esin, S., et al. (1996) Different Percentages of Peripheral Blood ‡ T Cells in Healthy Individuals from Different Areas of the World, Scand J Immunol.
Flament, H., et al. (2021) ‘Outcome of SARS-CoV-2 infection is linked to MAIT cell activation and cytotoxicity’, Nature Immunology, 22(3), pp. 322-335. doi: 10.1038/s41590-021-00870-z.
Folegatti, P. M., et al. (2020) ‘Safety and immunogenicity of the ChAdOxI nCoV-19 vaccine against SARS-CoV-2: a preliminary report of a phase 1/2, single-blind, randomised controlled trial’, The Lancet, 396(10249), pp. 467-478. doi: 10.1016/S0140-6736(20)31604-4.
Fonseca, S., et al. (2020) ‘Human Peripheral Blood Gamma Delta T Cells: Report on a Series of Healthy Caucasian Portuguese Adults and Comprehensive Review of the Literature’, Cells, 9(3), p. 729. doi: 10.3390/cells9030729.
Galson, J. D., et al. (no date) ‘Deep Sequencing of B Cell Receptor Repertoires From COVID-19 Patients Reveals Strong Convergent Immune Signatures’. doi: 10.3389/fimmu.2020.605170.
Goodwin, K., et al. (2006) ‘Antibody response to influenza vaccination in the elderly: A quantitative review’, Vaccine, 24(8), pp. 1159-1169. doi: 10.1016/j.vaccine.2005.08.105.
Grifoni, A., et al. (2020) ‘Targets of T Cell Responses to SARS-CoV-2 Coronavirus in Humans with COVID-19 Disease and Unexposed Individuals.’, Cell, 181(7), pp. 1489-1501.e15. doi: 10.1016/j.cell.2020.05.015.
Hansen, C. H., et al. (2021) ‘Assessment of protection against reinfection with SARS-CoV-2 among 4 million PCR-tested individuals in Denmark in 2020: a population-level observational study.’, Lancet (London, England), 0(0). doi: 10.1016/S0140-6736(21)00575-4.
Hayday, A. C. (2019) ‘γδ T Cell Update: Adaptate Orchestrators of Immune Surveillance’, The Journal of Immunology, 203(2), pp. 311-320. doi: 10.4049/jimmunol.1800934.
Hayday, A. C., S et al. (1985) ‘Structure, organization, and somatic rearrangement of T cell gamma genes’, Cell, 40(2), pp. 259-269. doi: 10.1016/0092-8674(85)90140-0.
Hayday, A. C. and Vantourout, P. (2020) ‘The Innate Biologies of Adaptive Antigen Receptors’, pp. 487-510.
Huang, H., Wang, C., Rubelt, F., Scriba, T. J. and Davis, M. M. (2020) ‘Analyzing the Mycobacterium tuberculosis immune response by T-cell receptor clustering with GLIPH2 and genome-wide antigen screening’, Nature Biotechnology, 38(10), pp. 1194-1202. doi: 10.1038/s41587-020-0505-4.
Hunter, S., et al. (2018) ‘Human liver infiltrating γδ T cells are composed of clonally expanded circulating and tissue-resident populations’, Journal of Hepatology, 69(3), pp. 654-665. doi: 10.1016/j.jhep.2018.05.007.
Jackson, L. A., et al. (2020) ‘An mRNA Vaccine against SARS-CoV-2 Preliminary Report’, New England Journal of Medicine, 383(20), pp. 1920-1931. doi: 10.1056/nejmoa2022483.
Jameson, J. M., et al. (2010) ‘A role for the mevalonate pathway in the induction of subtype cross-reactive immunity to influenza A virus by human γδ T lymphocytes’, Cellular Immunology, 264(1), pp. 71-77. doi: 10.1016/j.cellimm.2010.04.013.
Jean, C. M., et al. (2007) ‘Risk factors for West Nile virus neuroinvasive disease, California, 2005’, Emerging Infectious Diseases, 13(12), pp. 1918-1920. doi: 10.3201/eid1312.061265.
Joint Committee on Vaccination and Immunisation: advice on priority groups for COVID-19 vaccination, 30 Dec. 2020—GOV.UK (2020). Available at: https://www.gov.uk/government/publications/priority-groups-for-coronavirus-covid-19-vaccination-advice-from-the-jcvi-30-december-2020/joint-committee-on-vaccination-and-immunisation-advice-on-priority-groups-for-covid-19-vaccination-30-december-2020#refer (Accessed: 23 Mar. 2021).
Jouan, Y., et al. (2020) ‘Phenotypical and functional alteration of unconventional T cells in severe COVID-19 patients’, Journal of Experimental Medicine, 217(12). doi: 10.1084/jem.20200872.
Laing, A. G., et al. (2020) ‘A dynamic COVID-19 immune signature includes associations with poor prognosis’, Nature Medicine, 26(10), pp. 1623-1635. doi: 10.1038/s41591-020-1038-6.
Lavezzo, E., et al. (2020) ‘Suppression of a SARS-CoV-2 outbreak in the Italian municipality of Vo”, Nature, 584(7821), pp. 425-429. doi: 10.1038/s41586-020-2488-1.
Li, H. M., et al. (2016) ‘TCR repertoire of CD4+ and CD8+ T cells is distinct in richness, distribution, and CDR3 amino acid composition’, Journal of Leukocyte Biology, 99(3), pp. 505-513. doi: 10.1189/jlb.6a0215-071rr.
Li, H. and Pauza, C. D. (2011) ‘HIV envelope-mediated, CCR5/α4β7-dependent killing of CD4-negative γδ T cells which are lost during progression to AIDS.’, Blood, 118(22), pp. 5824-31. doi: 10.1182/blood-2011-05-356535.
Lucas, C., et al. (2020) Kinetics of antibody responses dictate COVID-19 outcome, medRxiv. medRxiv. doi: 10.1101/2020.12.18.20248331.
Mathew, D., et al. (2020) ‘Deep immune profiling of COVID-19 patients reveals distinct immunotypes with therapeutic implications’, Science, 369(6508). doi: 10.1126/SCIENCE.ABC8511.
Nielsen, S. C. A., et al. (no date) ‘B cell clonal expansion and convergent antibody responses to SARS-CoV-2’. doi: 10.21203/rs.3.rs-27220/v1.
Nielsen, S. C. A., et al. (2020) ‘Human B Cell Clonal Expansion and Convergent Antibody Responses to SARS-CoV-2’, Cell Host and Microbe, 28(4), pp. 516-525.e5. doi: 10.1016/j.chom.2020.09.002.
O'Driscoll, M., et al. (2021) ‘Age-specific mortality and immunity patterns of SARS-CoV-2’, Nature, 590(7844), pp. 140-145. doi: 10.1038/s41586-020-2918-0.
Onyema, O. O., et al. (2012) ‘Cellular aging and senescence characteristics of human T-lymphocytes’, Biogerontology, 13(2), pp. 169-181. doi: 10.1007/s10522-011-9366-z.
Peiris, J. S. M., et al. (2003) ‘Clinical progression and viral load in a community outbreak of coronavirus-associated SARS pneumonia: A prospective study’, Lancet, 361(9371), pp. 1767-1772. doi: 10.1016/S0140-6736(03)13412-5.
Piroth, L., et al. (2021) ‘Comparison of the characteristics, morbidity, and mortality of COVID-19 and seasonal influenza: a nationwide, population-based retrospective cohort study’, The Lancet Respiratory Medicine, 9(3), pp. 251-259. doi: 10.1016/52213-2600(20)30527-0.
Poccia, F., et al. (2006) ‘Anti-severe acute respiratory syndrome coronavirus immune responses: The role played by Vγ9Vδ2 T cells’, Journal of Infectious Diseases, 193(9), pp. 1244-1249. doi: 10.1086/502975.
Poccia, F., et al. (1996) ‘Peripheral V gamma 9/V delta 2 T cell deletion and anergy to nonpeptidic mycobacterial antigens in asymptomatic HIV-1-infected persons.’, The Journal of Immunology, 157(1).
Porritt, R. A., et al. (2021) ‘HLA class I-associated expansion of TRBV11-2 T cells in Multisystem Inflammatory Syndrome in Children.’, The Journal of clinical investigation. doi: 10.1172/JCI146614.
Qi, Q., et al. (2014) ‘Diversity and clonal selection in the human T-cell repertoire’, Proceedings of the National Academy of Sciences of the United States of America, 111(36), pp. 13139-13144. doi: 10.1073/pnas.1409155111.
Ravens, S., et al. (2017) ‘Human γδ T cells are quickly reconstituted after stem-cell transplantation and show adaptive clonal expansion in response to viral infection’, Nature Immunology, 18(4), pp. 393-401. doi: 10.1038/ni.3686.
Raybould, M. I. J., et al. (2020) ‘CoV-AbDab: the coronavirus antibody database’, Bioinformatics. Edited by J. Wren. doi: 10.1093/bioinformatics/btaa739.
Rijkers, G., Vervenne, T. and van der Pol, P. (2020) ‘More bricks in the wall against SARS-CoV-2 infection: involvement of 7962 T cells’, Cellular and Molecular Immunology, 17(7), pp. 771-772. doi: 10.1038/s41423-020-0473-0.
Robbiani, D. F., et al. (2020) ‘Convergent antibody responses to SARS-CoV-2 in convalescent individuals’, Nature, 584(7821), pp. 437-442. doi: 10.1038/s41586-020-2456-9.
Rutishauser, T., et al. (2020) ‘ Activation of TCR Vδ1+ and Vδ1−Vδ2−γδ T Cells upon Controlled Infection with Plasmodium falciparum in Tanzanian Volunteers’, The Journal of Immunology, 204(1), pp. 180-191. doi: 10.4049/jimmunol.1900669.
Sahin, U., et al. (2020) ‘COVID-19 vaccine BNT162b1 elicits human antibody and TH1 T cell responses’, Nature, 586(7830), pp. 594-599. doi: 10.1038/s41586-020-2814-7.
Schultheiρ, C., et al. (2020) ‘Next-Generation Sequencing of T and B Cell Receptor Repertoires from COVID-19 Patients Showed Signatures Associated with Severity of Disease’, Immunity, 53(2), pp. 442-455.e4. doi: 10.1016/j.immuni.2020.06.024.
Shen, L., et al. (2020) ‘Delayed specific IgM antibody responses observed among COVID-19 patients with severe progression’, Emerging Microbes & Infections, 9(1), pp. 1096-1101. doi: 10.1080/22221751.2020.1766382.
Sherwood, A. M., et al. (2011) ‘Deep Sequencing of the Human TCR and TCR Repertoires Suggests that TCR Rearranges After and T Cell Commitment’, Science Translational Medicine, 3(90), pp. 90ra61-90ra61. doi: 10.1126/scitranslmed.3002536.
Shomuradova, A. S., et al. (2020) ‘SARS-CoV-2 Epitopes Are Recognized by a Public and Diverse Repertoire of Human T Cell Receptors’, Immunity, 53(6), pp. 1245-1257.e5. doi: 10.1016/j.immuni.2020.11.004.
Sokal, A., et al. (2021) ‘Maturation and persistence of the anti-SARS-CoV-2 memory B cell response’, Cell, 184(5), pp. 1201-1213.e14. doi: 10.1016/j.cell.2021.01.050.
Song, J. W., et al. (2020) ‘Immunological and inflammatory profiles in mild and severe cases of COVID-19’, Nature Communications, 11(1), p. 3410. doi: 10.1038/s41467-020-17240-2.
Tabata, S., et al. (2020) ‘Clinical characteristics of COVID-19 in 104 people with SARS-CoV-2 infection on the Diamond Princess cruise ship: a retrospective analysis’, The Lancet Infectious Diseases, 20(9), pp. 1043-1050. doi: 10.1016/S1473-3099(20)30482-5.
Tyler, C. J., et al. (2015) ‘Human Vγ9/Vδ2 T cells: Innate adaptors of the immune system’, Cellular Immunology, 296(1), pp. 10-21. doi: 10.1016/j.cellimm.2015.01.008.
Verity, R., et al. (2020) ‘Estimates of the severity of coronavirus disease 2019: a model-based analysis’, The Lancet Infectious Diseases, 20(6), pp. 669-677. doi: 10.1016/51473-3099(20)30243-7.
Wilk, A. J., et al. (2020) ‘A single-cell atlas of the peripheral immune response in patients with severe COVID-19’, Nature Medicine, 26(7), pp. 1070-1076. doi: 10.1038/s41591-020-0944-y.
Willcox, B. E. and Willcox, C. R. (2019) ‘γδ TCR ligands: the quest to solve a 500-million-year-old mystery’, Nature Immunology, 20(2), pp. 121-128. doi: 10.1038/s41590-018-0304-y.
World Health Organization (2020) ‘Weekly Epidemiological Update on COVID-19’, World Health Organization, (3 November), p. 1;4.
Yamashita, S., T et al. (2003) ‘Recognition mechanism of non-peptide antigens by human γδ T cells’, International Immunology, 15(11), pp. 1301-1307. doi: 10.1093/intimm/dxg129.
Zhang, N. and Bevan, M. J. (2011) ‘CD8+ T Cells: Foot Soldiers of the Immune System’, Immunity. Elsevier, pp. 161-168. doi: 10.1016/j.immuni.2011.07.010.
Zheng, M., et al. (2020) ‘Functional exhaustion of antiviral lymphocytes in COVID-19 patients’, Cellular and Molecular Immunology, 17(5), pp. 533-535. doi: 10.1038/s41423-020-0402-2.

### ADDITIONAL EMBODIMENTS

Additional Embodiment 1. A method of determining disease state in a subject by determining an immune cell repertoire by detecting immune gene sequences in the cells by a method comprising:
a) contacting a sample containing a subject's immune cells with a plurality of immune cell receptor V gene specific primers, each primer including from 5′ to 3′: [5′-Phos], [SPLINT1], [BARCODE], and [V], wherein: [5′-Phos] is a 5′ phosphate; [SPLINT] is a first adaptor sequence; [BARCODE] is a unique molecular identifier barcode (UMI); and [V] is a sequence capable of hybridizing to an immune cell receptor V gene;
b) hybridizing and extending the V gene specific primers to form a plurality of first double-stranded primer extension products;
c) contacting the sample with an exonuclease to remove unhybridized V gene specific primers from the first double stranded primer extension products;
d) contacting the sample with a plurality of immune cell receptor J gene specific primers, each primer including from 5′ to 3′: [5′-Phos], [SPLINT2], and [J], wherein: [5′-Phos] is a 5′ phosphate; [SPLINT2] is a second adaptor sequence; and [J] is a sequence capable of hybridizing to an immune cell receptor J gene; and further contacting the sample with a first universal primer capable of hybridizing to the first adaptor sequence;
e) hybridizing and extending the J gene specific primers and the first universal primer to form a plurality of second double-stranded primer extension products;
f) contacting the sample with an exonuclease to remove unhybridized J gene specific primers and first universal primer from the second double-stranded primer extension products;
g) contacting the sample with first and second universal primers capable of hybridizing to the first and second adaptor sequences;
h) amplifying the plurality of second double-stranded primer extension products;
i) sequencing the amplified products to determine immune gene sequences, wherein a plurality of the gene sequences, each sequence associated with a UMI represents an immune cell repertoire;
j) comparing the immune cell repertoire, wherein a change in representation of an immune cell type in the patient's profile indicates a disease state.

Additional Embodiment 2. A method of simultaneously determining a repertoire of T-cells and B-cells in a subject by detecting immune gene sequences in the T-cells and B-cells by a method comprising:
a) contacting a sample containing a subject's immune cells with a plurality of immune cell receptor V gene specific primers, each primer including from 5′ to 3′: [5′-Phos], [SPLINT1], [BARCODE], and [V], wherein: [5′-Phos] is a 5′ phosphate; [SPLINT] is a first adaptor sequence; [BARCODE] is a unique molecular identifier barcode; and [V] is a sequence capable of hybridizing to an immune cell receptor V gene;
b) hybridizing and extending the V gene specific primers to form a plurality of first double-stranded primer extension products;
c) contacting the sample with an exonuclease to remove unhybridized V gene specific primers from the first double stranded primer extension products;
d) contacting the sample with a plurality of immune cell receptor J gene specific primers, each primer including from 5′ to 3′: [5′-Phos], [SPLINT2], and [J], wherein: [5′-Phos] is a 5′ phosphate; [SPLINT2] is a second adaptor sequence; and [J] is a sequence capable of hybridizing to an immune cell receptor J gene; and further contacting the sample with a first universal primer capable of hybridizing to the first adaptor sequence;
e) hybridizing and extending the J gene specific primers and the first universal primer to form a plurality of second double-stranded primer extension products;
f) contacting the sample with an exonuclease to remove unhybridized J gene specific primers and first universal primer from the second double-stranded primer extension products;
g) contacting the sample with first and second universal primers capable of hybridizing to the first and second adaptor sequences;
h) amplifying the plurality of second double-stranded primer extension products;
i) sequencing the amplified products to determine immune gene sequences;
j) grouping the immune gene sequences having the same unique molecular identified barcode (UMI) and the same complementarity determining region 3 (CDR3) into groups, each group representing a single immune cell;
k) determining a consensus within the groups thereby determining the patient's repertoire of immune cells.

Additional Embodiment 3. The method of additional embodiment 2, wherein only immune gene sequences representing productive rearrangements are used in determining the patient's repertoire of immune cells.

Additional Embodiment 4. The method of additional embodiment 2, wherein at least 20,000 unique CDR3 sequences are identified in step j).

Additional Embodiment 5. The method of additional embodiment 2, wherein the V gene primers comprise a combination of Vα, Vβ, Vγ, Vδ, VH (immunoglobulin) gene primers.

Additional Embodiment 6. The method of additional embodiment 2, wherein the J gene primers comprise a combination of Jα, Jβ, Jγ, Jδ, JH (immunoglobulin) gene primers.

Additional Embodiment 7. The method of additional embodiment 2, wherein the sample comprising patient's immune cells is obtained by separating cells from blood plasma.

Additional Embodiment 8. The method of additional embodiment 7, wherein the sample comprising patient's immune cells is obtained by capturing CD4+ cells.

Additional Embodiment 9. The method of additional embodiment 7, wherein the sample comprising patient's immune cells comprises CD4+ cells and CD8+ cells.

Additional Embodiment 10. The method of additional embodiment 2, further comprising measuring immune cell repertoire diversity.

Additional Embodiment 11. The method of additional embodiment 2, further comprising measuring immune cell repertoire focusing.

Additional Embodiment 12. A method of measuring a subject's immune response to an infection by measuring the subject's immune cell repertoire by the method of additional embodiment 11, wherein the presence of focusing indicates immune response to the infection.

Additional Embodiment 13. A method of determining relative numbers of T-cells and B-cells in a subject comprising measuring the subject's immune cell repertoire by the method of additional embodiment 2, and determining the ratio of the number of T-cell receptor (TCR) sequences to the number of B-cell receptor (BCR) sequences.

Additional Embodiment 14. The method of additional embodiment 13, wherein the TCR sequences consist of TRD and TRB sequences.

Additional Embodiment 15. The method of additional embodiment 13, wherein the BCR sequences consist of IgH sequences.

Additional Embodiment 16. A method of detecting an immune reaction to a superantigen in a subject comprising measuring the subject's immune cell repertoire by the method of additional embodiment 2, wherein an increased number of Vβ T-cells indicates immune response to a superantigen.

Additional Embodiment 17. The method of additional embodiment 16, wherein the increase in measured by normalizing the number of Vβ T-cells against the amount of DNA in the sample.

Additional Embodiment 18. The method of additional embodiment 16, wherein the increase in measured by normalizing the number of Vβ T-cells against the number of cells in the sample.

Additional Embodiment 19. A method of assessing prevalence of mucosal associated invariant T-cells (MAIT) in a subject comprising measuring the subject's immune cell repertoire by the method of additional embodiment 2, and determining a ratio of unique Vp sequences associated with MAIT to unique Vβ sequences associated with non-MAIT T-cells, thereby determining the prevalence of MAIT in the subject.

Additional Embodiment 20. A method of assessing prevalence of mucosal associated invariant T-cells (MAIT) in a test subject comprising measuring by the method of additional embodiment 2, the immune cell repertoires in a test subject and in a control subject, and comparing the numbers of unique Vβ sequences associated with MAIT in the subject and in the control subject, thereby determining the prevalence of MAIT in the test subject.

Additional Embodiment 21. The method of additional embodiment 20, wherein the increase in measured by normalizing the number of Vβ T-cells against the amount of DNA in the sample.

Additional Embodiment 22. The method of additional embodiment 20, wherein the increase in measured by normalizing the number of Vβ T-cells against the number of cells in the sample.

Additional Embodiment 23. A method of determining pathogen-specific immune sequences comprising: measuring by the method of additional embodiment 2, and comparing immune cell repertoire in one or more subjects infected with a pathogen and one or more control subjects, determining at least one immune cell present in more than one infected subject but not in the control subjects, thereby determining pathogen-specific immune cell sequences.

Additional Embodiment 24. The method of additional embodiment 23, wherein the pathogen-specific immune sequences include one or more TCRs.

Additional Embodiment 25. The method of additional embodiment 23, wherein the pathogen-specific immune sequences include one or more BCRs.

Additional Embodiment 26. A method of detecting immune cell loss in a subject comprising measuring by the method of additional embodiment 2 and comparing immune cell repertoire in a subject and one or more control subjects, determining immune sequences present at a reduced level in the subject compared to the control subjects thereby detecting immune cell loss.

Additional Embodiment 27. A method of determining pathogen-specific immune sequences comprising: determining a subject's immune cell repertoire by the method of embodiment 2, and comparing determined immune cell repertoire in one or more subjects infected with SARS-CoV-2 and one or more control subjects, determining at least one immune cell present in more than one infected subject but not in the control subjects, thereby determining pathogen-specific immune cell sequences.

Additional Embodiment 28. A method of detecting immune cell loss in a subject infected with SARS-CoV-2 comprising: determining a subject's immune cell repertoire by the method of embodiment 2, and comparing the determined immune cell repertoire in the subject and the control immune cell repertoires derived from one or more control subjects, identifying immune sequences present at a reduced level in the subject compared to the control subjects thereby detecting immune cell loss.

Additional Embodiment 29. A method of simultaneously characterizing antigen receptor repertoires of CD4+ and CD8+ αβ T cells, B cells, and Vδ2+ and Vδ1+ T cells in a sample derived from a subject infected with SARS-CoV-2 infection, comprising:
a) contacting a sample derived from the subject with a plurality of immune cell receptor V gene specific primers, each primer including from 5′ to 3′: [5′-Phos], [SPLINT1], [BARCODE], and [V], wherein: [5′-Phos] is a 5′ phosphate; [SPLINT] is a first adaptor sequence; [BARCODE] is a unique molecular identifier barcode (UMI); and [V] is a sequence capable of hybridizing to an immune cell receptor V gene in the sample;
b) hybridizing and extending the V gene specific primers to form a plurality of first double-stranded primer extension products;
c) contacting the sample with an exonuclease to remove unhybridized V gene specific primers from the first double stranded primer extension products;
d) contacting the sample with a plurality of immune cell receptor J gene specific primers, each primer including from 5′ to 3′: [5′-Phos], [SPLINT2], and [J], wherein: [5′-Phos] is a 5′ phosphate; [SPLINT2] is a second adaptor sequence; and [J] is a sequence capable of hybridizing to an immune cell receptor J gene; and further contacting the sample with a first universal primer capable of hybridizing to the first adaptor sequence;
e) hybridizing and extending the J gene specific primers and the first universal primer to form a plurality of second double-stranded primer extension products;
f) contacting the sample with an exonuclease to remove unhybridized J gene specific primers and first universal primer from the second double-stranded primer extension products;
g) contacting the sample with first and second universal primers capable of hybridizing to the first and second adaptor sequences;
h) amplifying the plurality of second double-stranded primer extension products;
i) sequencing the amplified products to determine a plurality of immune gene sequences associated with the CD4+ and CD8+ αβ T cells, the B cells, and the Vδ2+ and Vδ1+ T cells, wherein each determined immune gene sequence of the plurality of determined immune gene sequences that is associated with a UMI represents a different antigen receptor repertoire.

Additional Embodiment 30. A method of simultaneously enriching a sample for a plurality of different target polynucleotides, wherein the plurality of different target polynucleotides comprise immune gene sequences derived from αβ T cells, B cells, and Vδ2+ and Vδ1+ T cells, the method comprising:
a) contacting the sample with a plurality of immune cell receptor V gene specific primers, each primer including from 5′ to 3′: [5′-Phos], [SPLINT1], [BARCODE], and [V], wherein: [5′-Phos] is a 5′ phosphate; [SPLINT] is a first adaptor sequence; [BARCODE] is a unique molecular identifier barcode; and [V] is a sequence capable of hybridizing to an immune cell receptor V gene;
b) hybridizing and extending the V gene specific primers to form a plurality of first double-stranded primer extension products;
c) contacting the sample with an exonuclease to remove unhybridized V gene specific primers from the first double stranded primer extension products;
d) contacting the sample with a plurality of immune cell receptor J gene specific primers, each primer including from 5′ to 3′: [5′-Phos], [SPLINT2], and [J], wherein: [5′-Phos] is a 5′ phosphate; [SPLINT2] is a second adaptor sequence; and [J] is a sequence capable of hybridizing to an immune cell receptor J gene; and further contacting the sample with a first universal primer capable of hybridizing to the first adaptor sequence;
e) hybridizing and extending the J gene specific primers and the first universal primer to form a plurality of second double-stranded primer extension products;
f) contacting the sample with an exonuclease to remove unhybridized J gene specific primers and first universal primer from the second double-stranded primer extension products;
g) contacting the sample with first and second universal primers capable of hybridizing to the first and second adaptor sequences;
h) amplifying the plurality of second double-stranded primer extension products;
i) sequencing the amplified products to determine the immune gene sequences;
j) grouping the determined immune gene sequences having the same unique molecular identified barcode (UMI) and the same complementarity determining region 3 (CDR3) into groups, each group representing a single immune cell; and
k) determining a consensus within the groups thereby determining the repertoire of T-cells and B-cells in the sample.

1. A method of simultaneously determining a repertoire of T-cells and B-cells in a sample derived from a subject infected with SARS-CoV-2 by detecting immune gene sequences in the T-cells and B-cells by a method comprising:
a) contacting the sample with a plurality of immune cell receptor V gene specific primers, each primer including from 5′ to 3′: [5′-Phos], [SPLINT1], [BARCODE], and [V], wherein: [5′-Phos] is a 5′ phosphate; [SPLINT] is a first adaptor sequence; [BARCODE] is a unique molecular identifier barcode; and [V] is a sequence capable of hybridizing to an immune cell receptor V gene;
b) hybridizing and extending the V gene specific primers to form a plurality of first double-stranded primer extension products;
c) contacting the sample with an exonuclease to remove unhybridized V gene specific primers from the first double stranded primer extension products;
d) contacting the sample with a plurality of immune cell receptor J gene specific primers, each primer including from 5′ to 3′: [5′-Phos], [SPLINT2], and [J], wherein: [5′-Phos] is a 5′ phosphate; [SPLINT2] is a second adaptor sequence; and [J] is a sequence capable of hybridizing to an immune cell receptor J gene; and further contacting the sample with a first universal primer capable of hybridizing to the first adaptor sequence;
e) hybridizing and extending the J gene specific primers and the first universal primer to form a plurality of second double-stranded primer extension products;
f) contacting the sample with an exonuclease to remove unhybridized J gene specific primers and first universal primer from the second double-stranded primer extension products;
g) contacting the sample with first and second universal primers capable of hybridizing to the first and second adaptor sequences;
h) amplifying the plurality of second double-stranded primer extension products;
i) sequencing the amplified products to determine the immune gene sequences;
j) grouping the determined immune gene sequences having the same unique molecular identified barcode (UMI) and the same complementarity determining region 3 (CDR3) into groups, each group representing a single immune cell; and
k) determining a consensus within the groups thereby determining the repertoire of T-cells and B-cells in the sample.

2. The method of claim 1, wherein at least 20,000 unique CDR3 sequences are identified in step j), and wherein the V gene primers comprise a combination of VH (immunoglobulin) gene primers selected from the group consisting of Vα, Vβ, Vγ, and Vδ, gene primers.

3. The method of claim 1, wherein at least 20,000 unique CDR3 sequences are identified in step j), and wherein the J gene primers comprise a combination of JH (immunoglobulin) gene primers selected from the group consisting of Jα, Jβ, Jγ, and Jδ primers.

4. The method of claim 1, wherein only immune gene sequences representing productive rearrangements are used in determining the patient's repertoire of immune cells.

5. The method of claim 1, wherein the sample comprises cells separated from blood plasma.

6. The method of claim 5, wherein the sample comprises captured CD4+ cells, and optionally CD8+ cells.

7. The method of claim 1, wherein the repertoire of T-cells and B-cells comprises a CD4+ αβ T cell repertoire, a CD8+ αβ T cell repertoire, a B cell repertoire, a Vδ2+ T cell repertoire, and a Vδ1+ T cell repertoire.

8. The method of claim 1, wherein the immune gene sequences in the T-cells comprise T-cell receptor (TCR) sequences, further comprising measuring a quantity of the TCR sequences.

9. The method of claim 8, wherein the TCR sequences comprise TRD sequences, TRB sequences, or a combination of TRD and TRB sequences.

10. The method of claim 8, wherein the immune gene sequences in the B-cells comprise B-cell receptor (BCR) sequences, further comprising measuring a quantity of the BCR sequences.

11. The method of claim 10, further comprising determining a ratio of the measured quantity of the TCR sequence to the measured quantity of the BCR sequences.

12. The method of claim 1, further comprising measuring immune cell repertoire diversity.

13. The method of claim 1, further comprising measuring immune cell repertoire focusing.

14. The method of claim 1, wherein the hybridizing in steps b) and/or e) comprises one or more cycles of a step-wise temperature drop of two or more steps.

15. The method of claim 1, wherein the hybridizing in steps b) and/or e) comprises 20 cycles of temperature change from 60° C. to 57.5° C. and to 55° C.

16. A method of characterizing a subject's antigen receptor repertoires by simultaneously enriching a sample derived from the subject for a plurality of immune gene sequences, wherein the subject is infected with SARS-CoV-2 infection, comprising:
a) contacting a sample derived from the subject with a plurality of immune cell receptor V gene specific primers, each primer including from 5′ to 3′: [5′-Phos], [SPLINT1], [BARCODE], and [V], wherein: [5′-Phos] is a 5′ phosphate; [SPLINT] is a first adaptor sequence;
[BARCODE] is a unique molecular identifier barcode (UMI); and [V] is a sequence capable of hybridizing to an immune cell receptor V gene in the sample;
b) hybridizing and extending the V gene specific primers to form a plurality of first double-stranded primer extension products;
c) contacting the sample with an exonuclease to remove unhybridized V gene specific primers from the first double stranded primer extension products;
d) contacting the sample with a plurality of immune cell receptor J gene specific primers, each primer including from 5′ to 3′: [5′-Phos], [SPLINT2], and [J], wherein: [5′-Phos] is a 5′ phosphate; [SPLINT2] is a second adaptor sequence; and [J] is a sequence capable of hybridizing to an immune cell receptor J gene; and further contacting the sample with a first universal primer capable of hybridizing to the first adaptor sequence;
e) hybridizing and extending the J gene specific primers and the first universal primer to form a plurality of second double-stranded primer extension products;
f) contacting the sample with an exonuclease to remove unhybridized J gene specific primers and first universal primer from the second double-stranded primer extension products;
g) contacting the sample with first and second universal primers capable of hybridizing to the first and second adaptor sequences;
h) amplifying the plurality of second double-stranded primer extension products;
i) sequencing the amplified products to determine the plurality of immune gene sequences, wherein each determined immune gene sequence of the plurality of determined immune gene sequences that is associated with a UMI represents a different immune cell repertoire.

17. A method of determining disease state in a subject infected with SARS-CoV-2 by determining an immune cell repertoire by detecting immune gene sequences in the cells by a method comprising:
a) contacting a sample derived from the subject with a plurality of immune cell receptor V gene specific primers, each primer including from 5′ to 3′: [5′-Phos], [SPLINT1], [BARCODE], and [V], wherein: [5′-Phos] is a 5′ phosphate; [SPLINT] is a first adaptor sequence;
[BARCODE] is a unique molecular identifier barcode (UMI); and [V] is a sequence capable of hybridizing to an immune cell receptor V gene in the sample;
b) hybridizing and extending the V gene specific primers to form a plurality of first double-stranded primer extension products;
c) contacting the sample with an exonuclease to remove unhybridized V gene specific primers from the first double stranded primer extension products;
d) contacting the sample with a plurality of immune cell receptor J gene specific primers, each primer including from 5′ to 3′: [5′-Phos], [SPLINT2], and [J], wherein: [5′-Phos] is a 5′ phosphate; [SPLINT2] is a second adaptor sequence; and [J] is a sequence capable of hybridizing to an immune cell receptor J gene; and further contacting the sample with a first universal primer capable of hybridizing to the first adaptor sequence;
e) hybridizing and extending the J gene specific primers and the first universal primer to form a plurality of second double-stranded primer extension products;
f) contacting the sample with an exonuclease to remove unhybridized J gene specific primers and first universal primer from the second double-stranded primer extension products;
g) contacting the sample with first and second universal primers capable of hybridizing to the first and second adaptor sequences;
h) amplifying the plurality of second double-stranded primer extension products;
i) sequencing the amplified products to determine a plurality of immune gene sequences, wherein each determined immune gene sequence of the plurality of determined immune gene sequences that is associated with a UMI represents a different immune cell repertoire; and
j) comparing the different determined immune cell repertoires to control immune cell repertoire data, wherein a change in representation of an immune cell type in the subject's antigen receptor repertoire indicates a disease state.
